(19) United States
(12) Patent Application Publication
(10) Pub. No.: US 2026/0354962 A1
(43) Pub. Date: Nov. 12, 2026

(54) GRAFT

(71) Applicant: DSM IP Assets B.V., 6221 BE Maastricht (NL)

(72) Inventors: Wei CHENG, Echt (NL); Marc LINSSEN, Echt (NL)

(21) Appl. No.: 19/130,362

(22) PCT Filed: Nov. 16, 2023
(86) PCT No.: PCT/EP2023/082154

§ 371 Date: May 15, 2025

Related U.S. Application Data
(60) Provisional application No. 63/426,378, filed on Nov. 18, 2022.

(51) Int. Cl. A61F 2/07

(52) U.S. Cl. CPC A61F 2/07; A61F 2002/072; A61F 2210/0076; A61F 2240/001

(57) ABSTRACT

A graft includes a tube having an inner film layer, an outer film layer disposed radially outward of the inner film layer, and a foam material disposed radially outward of the inner film layer, the outer film layer covering a covered portion of the foam material that is disposed between the outer film layer and the inner film layer, the foam material further including an exposed portion that is not covered by the outer film layer.

### FIELD

The disclosure relates to grafts, methods of making grafts, and the use of such grafts to treat various medical conditions, such as aortic aneurysms or peripheral artery disease.

### BACKGROUND

Grafts are commonly used to treat diseased blood vessels and other tubular structures within the body. For example, a graft may be employed to reinforce or radially open blood vessels for the purpose of restoring or maintaining blood flow. Grafts may be implanted in the coronary, aortic and peripheral vasculature, neurovasculature, and in other bodily conduits such as the urinary tract, the bile duct, and the tracheo-bronchial tree.

Grafts typically comprise a metal stent covered by a fabric, film, and/or membrane. Various designs are possible for the metal stent, but they typically comprise a series of metal rings connected by metal bridges. The covering is typically a PTFE, an ePTFE (expanded PTFE) membrane, or a woven or knitted PET fabric.

Endovascular grafts require compressibility and flexibility to allow for proper positioning in the vasculature. Such grafts are either self-expandable or balloon expandable. A self-expandable graft expands upon removal of the constraint that keeps the graft from expanding. Balloon expandable grafts use a balloon to expand the graft. In either case, the graft must be compressible during delivery and positioning and expand to exert pressure on the vessel wall upon delivery. Nitinol is often a preferred material for a reinforcing element of such a graft due to its ability to withstand a significant amount of bending without permanent deformation.

A major complication of such grafts is endoleak, particularly in the case of treating thoracic or abdominal aortic aneurysms. Endoleak occurs when blood finds a way around the graft, such as into an aneurysm, a potentially life-threatening condition. Endoleak may result from insufficient anchoring of the graft against the vessel wall or migration of the stent. Such problems may be more prevalent in sutureless grafts, which are preferred in the industry for their simplicity and minimal invasiveness.

Various techniques have been implemented to reduce endoleak without the use of sutures. One method is to increase the pressure exerted by the graft against the vessel wall. However, an improved graft that minimizes endoleak would be desirable.

### SUMMARY

In accordance with the invention, a graft comprises a foam material that is exposed at one or more locations and covered at one or more locations along the graft. The foam material may allow for tighter apposition against the vessel wall at least at the one or more exposed portions. In an embodiment, the foam comprises a porous outer surface at the exposed portions. The pores may facilitate local thrombus formation and/or tissue ingrowth that may minimize or even prevent endoleak due to the resulting improved anchoring of the graft against the vessel wall.

Accordingly, in an embodiment, a graft comprises a tube comprising an inner film layer, an outer film layer, and a foam material between the inner film and the outer film, wherein the tube comprises one or more covered portions, wherein the foam material is covered by the outer film and inner film, and one or more exposed portions, wherein the foam is not covered by the outer film but may be covered by the inner film. The graft may further comprise a reinforcing element, such as a stent, and/or additional film layers.

In accordance with a further aspect of the invention, a method is provided to form such a graft having one or more exposed portions of the foam. The method may allow for flexibility of the exposed portion along the tube of the graft. For example, in an embodiment the graft is fenestrated and an exposed portion is present at the connection between a branch and the main body of the graft.

In an embodiment, a method of forming a graft comprises the steps of:
a. providing a first film layer present on a mandrel;
b. placing a foam on the first film such that the foam is over the first film layer at a first location;
c. placing a second film layer on the foam and first film, thereby creating a covered portion, wherein the foam is covered by the first film and the second film, and an exposed portion, wherein the foam is not covered by at least the second film; and
d. thermally bonding the first and second film layers together to connect the first film layer, the foam material, and the second film layer together, thereby forming a tube.

The first film layer may be the inner film layer of a tube or a mid-film layer of a tube. The second film may be a mid-film layer of the tube or an outer film layer of the tube. By placing or providing a film on a mandrel, it does not mean that the film must be in contact with the mandrel. Rather, the film may be in contact with the mandrel, other components of the graft (such as a stent), or components that aid in formation of the graft with the method, such as a release layer.

The use of an ultra-high molecular weight polyethylene (UHMWPE) membrane as a film may provide certain advantages to the graft. Principally, a UHMWPE membrane allows for the disclosed method of forming a graft to be performed at desirable process conditions, enabling use of certain foams that would thermally degrade if the method was performed at the temperatures needed to soften and laminate other graft materials, such as ePTFE.

The inventions described herein may offer numerous improvements over prior art grafts including reduced endoleak, reduced migration, improved durability, improved tissue integration, simplicity or cost of manufacturing, cost, or other advantages.

### BRIEF DESCRIPTION OF FIGURES

FIG. 1 is a side view of one embodiment of a graft according to aspects of the disclosure;

FIG. 2 is a perspective view of a portion of the graft of FIG. 1;

FIG. 3 is a schematic perspective view of a portion of one embodiment of a method of manufacturing the graft of FIG. 1;

FIG. 4 is a schematic cross-section view showing a portion of the method of manufacturing the graft of FIG. 3;

FIG. 5 is a side view of another embodiment of a graft according to aspects of the disclosure;

FIG. 6 is a schematic side view of a portion of one embodiment of a method of manufacturing the graft of FIG. 5;

FIG. 7 is a schematic exploded view of a portion of the graft of FIG. 1;

FIG. 8 is a schematic cross-section view of one embodiment of a connection arrangement between a foam material and a film layer according to aspects of the disclosure;

FIG. 8A is a schematic cross-section view of another embodiment of a connection arrangement between a foam material and a film layer according to aspects of the disclosure;

FIG. 9 is a schematic cross-section view of another embodiment of a connection arrangement between a foam material and a film layer according to aspects of the disclosure;

FIG. 10 is a schematic cross-section view of another embodiment of a connection arrangement between a foam material and a film layer according to aspects of the disclosure;

FIG. 11 is a schematic cross-section view of another embodiment of a connection arrangement between a foam material and a film layer according to aspects of the disclosure;

FIG. 12 is a schematic exploded view of a portion of another embodiment of a graft according to aspects of the disclosure;

FIG. 13 is a schematic exploded view of a portion of another embodiment of a graft according to aspects of the disclosure;

FIG. 14 is a schematic exploded view of a portion of another embodiment of a graft according to aspects of the disclosure;

FIG. 15 is a schematic exploded view of a portion of another embodiment of a graft according to aspects of the disclosure;

FIG. 16 is a side view of another embodiment of a graft according to aspects of the disclosure;

FIG. 17 is a side view of another embodiment of a graft according to aspects of the disclosure;

FIG. 18 is a side view of another embodiment of a graft according to aspects of the disclosure;

FIG. 19 is a magnified view of a portion of the graft of FIG. 18;

FIG. 20 is a side view of a stent and a foam material for use with another embodiment of a graft according to aspects of the disclosure;

FIG. 21 is a perspective view of the stent and the foam material of FIG. 20;

FIG. 22 is a schematic side view of another embodiment of a graft according to aspects of the disclosure;

FIG. 23 is a schematic side view of another embodiment of a graft according to aspects of FIG. 24 is a schematic side view of another embodiment of a graft according to aspects of the disclosure;

FIG. 25 is a schematic side view of another embodiment of a graft according to aspects of the disclosure;

FIG. 26 is a side view of another embodiment of a graft according to aspects of the disclosure; and

FIG. 27 is a schematic side view of another embodiment of a graft according to aspects of the disclosure.

### DETAILED DESCRIPTION

While this invention is susceptible of embodiments in many different forms, there are shown in the drawings and will herein be described in detail example embodiments of the invention with the understanding that the present disclosure is to be considered as an exemplification of the principles of the invention and is not intended to limit the broad aspect of the invention to the embodiments illustrated. In the following description of various example structures according to the invention, reference is made to the accompanying drawings, which form a part hereof, and in which are shown by way of illustration various example devices, systems, and environments in which aspects of the invention may be practiced. It is to be understood that other specific arrangements of parts, example devices, systems, and environments may be utilized and structural and functional modifications may be made without departing from the scope of the present invention.

Various embodiments of the invention relate to a graft 10 that includes a tube 12 formed of a plurality of layers that define a tubular structure that is elongated between opposed first and second ends 13, 14 and has a cylindrical inner surface 15 defining a central passage 16 and a cylindrical outer surface 17 opposite the inner surface 15. The plurality of layers include a foam material 40 that is at least partially exposed on the outer surface of the tube 12. The plurality of layers may include at least one film layer 20, 22, 24 and/or a reinforcing element 30 that define the tubular structure of the tube 12. The foam material 40 may be disposed to cover one or more inner layers disposed radially inward of the foam material 40 and may include a covered portion 42 that is covered by one or more outer layers, leaving an exposed portion 44 that is not covered by the outer layer(s).

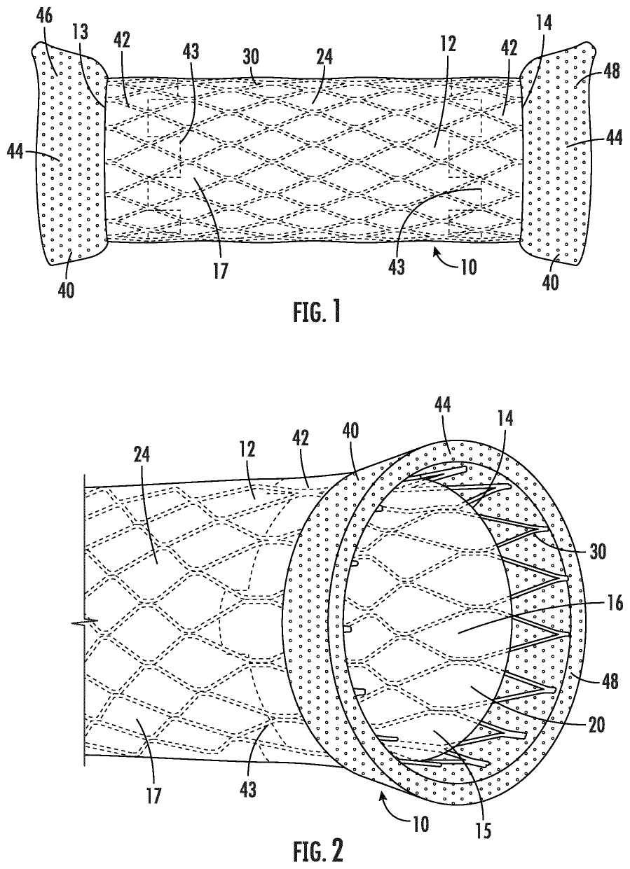

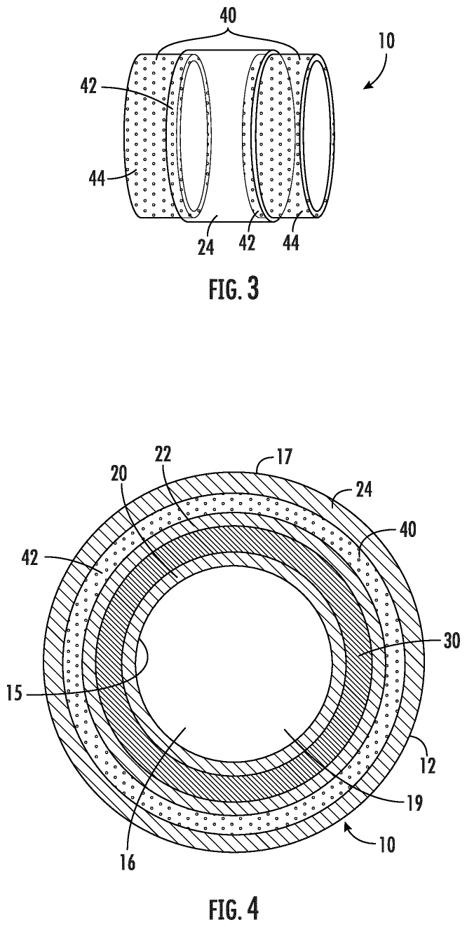

FIGS. 1-2 illustrate one embodiment of a graft 10, and FIGS. 3-4 illustrate one embodiment of a method of production of the graft 10 of FIGS. 1-2. In this embodiment, the graft 10 comprises a tube 12 formed of three film layers, including an inner film layer 20, a middle film layer 22, and an outer film layer 24, as well as a reinforcing element 30 and a foam material 40. The outer film layer 24 is disposed radially outward of the inner and middle film layers 20, 22, and the middle film layer 22 is disposed radially outward of the inner film layer 20 and between the inner and outer film layers 20, 24. FIG. 7 schematically illustrates the layering of the layers forming the tube 12 in FIGS. 1-4. The reinforcing element 30 in this embodiment is in the form of a stent as described herein that extends the entire length of the tube 12, from the first end 13 to the second end 14. As seen in FIG. 2, the reinforcing element 30 extends axially beyond the ends of the film layers 20, 22, 24 at both ends 13, 14 of the tube 12. The reinforcing element 30 is disposed between the inner film layer 20 and the middle film layer 22 and is connected to both the inner and middle film layers 20,22.

The foam material 40 in the embodiment of FIGS. 1-4 includes a first foam member 46 at the first end 13 of the tube 12 and a second foam member 48 at the second end 14 of the tube 12. Each foam member 46, 48 has a covered portion 42 that is covered by the outer film layer 24, which is positioned between and connected to or otherwise engaged by the outer film layer 24 and the middle film layer 22. Additionally, each foam member 46, 48 has an exposed portion 44 that is positioned axially outward of the covered portion 42, such that the covered portion 42 extends axially inward from the exposed portion 44. The covered portions 42 in the graft 10 of FIGS. 1-4 have a stepped distal edge 43 to increase contact between the foam member 46, 48 and the adjacent film layers 22, 24 and thereby increase retention force between the foam member 46, 48 and the film layers 22, 24. The foam members 46, 48 are configured as annular collars that extend around the entire circumference of the outer surface 17 of the tube 12, and each foam member 46, 48 is formed of a single piece of the foam material 40 wrapped around the tube 12. The foam members 46, 48 in this embodiment are positioned such that the exposed portions 44 extend axially beyond the ends of the film layers 20, 22, 24 at both ends 13, 14 of the tube 12, as seen in FIGS. 2 and 3.

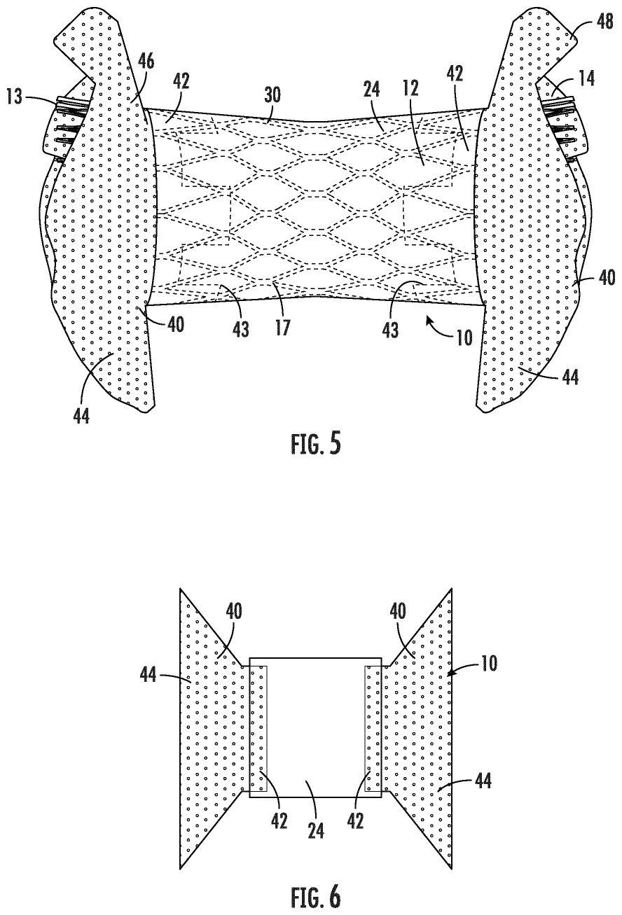

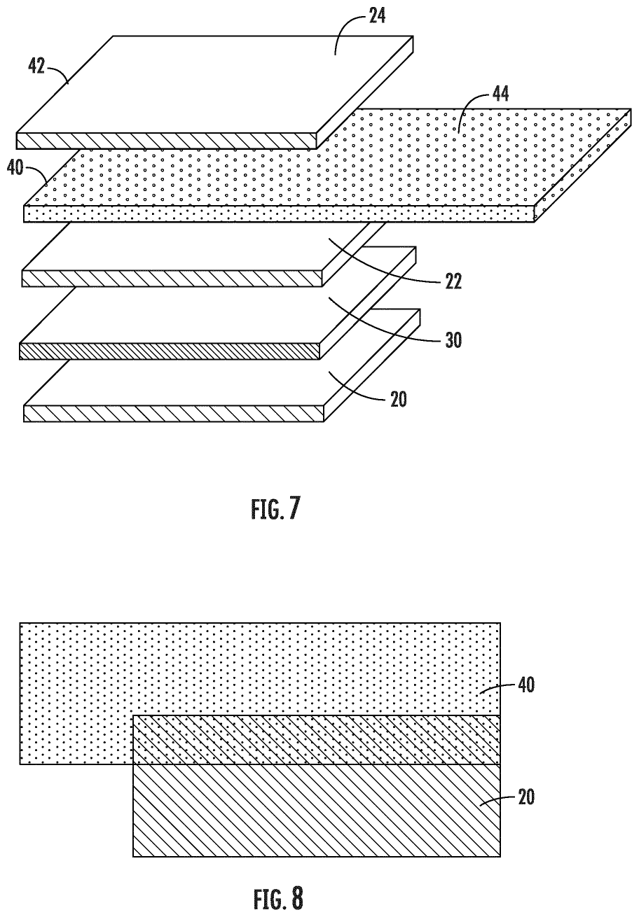

FIGS. 5-6 illustrate another embodiment of a graft 10 comprising a tube 12 that includes a structure of layers that is similar to the graft 10 in FIGS. 1-4, having an inner film layer 20, a reinforcing element 30, a middle film layer 22, a foam material 40, and an outer film layer 24 in layered order. In the embodiment of FIGS. 5-6, the foam members 46, 48 of the foam material 40 have reduced thickness relative to the foam members 46, 48 of the tube 12 of FIGS. 1-4, and the foam members 46, 48 in FIGS. 5-6 are configured as annular or frusto-conical collars having a flared configuration that permits the foam members 46, 48 to expand radially at the distal ends thereof. The graft of FIGS. 5-6 is otherwise similar to the embodiment in FIGS. 1-4.

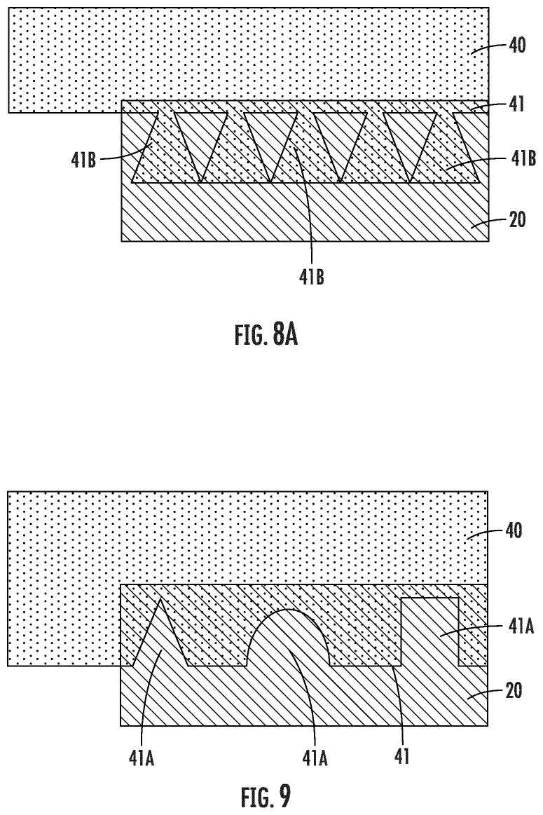

FIGS. 8-11 illustrate various connection configurations for overlapping connection between a foam material 40 and two or more film layers 20, 22, 24 as described herein, which may be used in connection with the embodiments of FIGS. 1-4 and/or 5-6, or any other embodiment described herein. While FIGS. 8-11 each illustrate a single member or portion of foam material 40 and a single film layer 24, the connection configurations in FIGS. 8-11 can be used to connect multiple components together, e.g., two film layers to one foam member.

FIG. 8 illustrates a foam material 40 in surface-to-surface contact with a film layer 20, in which a portion of the film layer 24 infiltrates the foam material 40 during heating in the manufacturing process as described herein. This infiltration increases the retention strength between the components. In one embodiment, the overlap distance (axially) between the foam material 40 and the film 24 in this configuration is at least 2-5 mm.

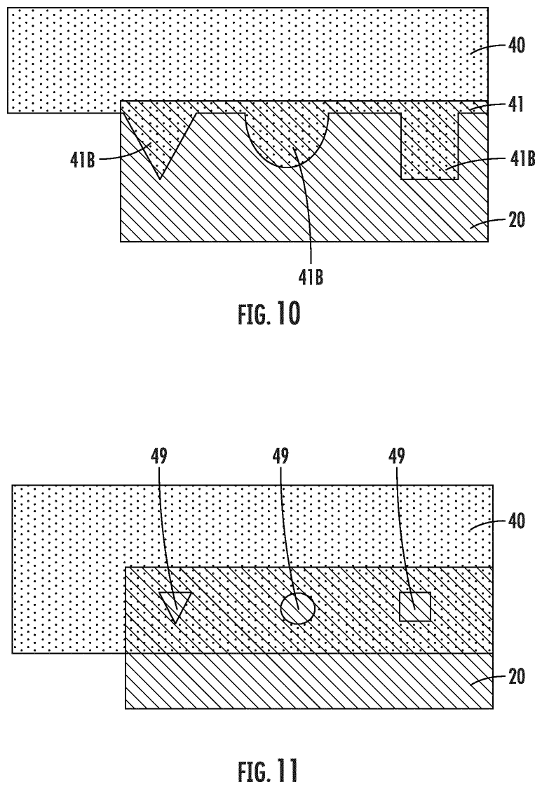

FIGS. 8A, 9, and 10 illustrate a foam material 40 with a shaped edge 41 in surface-to-surface contact with a film layer 24. The shaped edge 41 of the foam material 40 in FIG. 8A includes a plurality of cutouts or recesses 41C extending inward from the edge 41 of the foam material 40, which are penetrated by the film layer 24 after heating in the manufacturing process as described herein. The cutouts 41C in FIG. 8A are triangular in shape, having a narrow width at the edge 41 and increasing in width as they extend axially. The shaped edge 41 of the foam material 40 in FIG. 9 includes a plurality of recesses 41A that are penetrated by the film layer 24 after heating in the manufacturing process as described herein. The shaped edge 41 of the foam material 40 in FIG. 10 includes a plurality of projections 41B that extend into the film layer 24 after heating in the manufacturing process as described herein. The engagement between the film layer 24 and the shaped edge 41 increases retention strength between the components. In one embodiment, the overlap distance (axially) between the foam material 40 and the film 24 in the configurations of FIGS. 9 and 10 is 3-7 mm, including 1-2 mm along a straight portion of the shaped edge 41 and 2-5 mm along the shaped portion of the shaped edge 41.

FIG. 11 illustrates a foam material 40 in surface-to-surface contact with a film layer 20, in which the foam material 40 includes holes 49 that are penetrated by the film layer 24 during heating in the manufacturing process as described herein. This infiltration increases the retention strength between the components. In one embodiment, the overlap distance (axially) between the foam material 40 and the film 24 in this configuration is at least 3-5 mm.

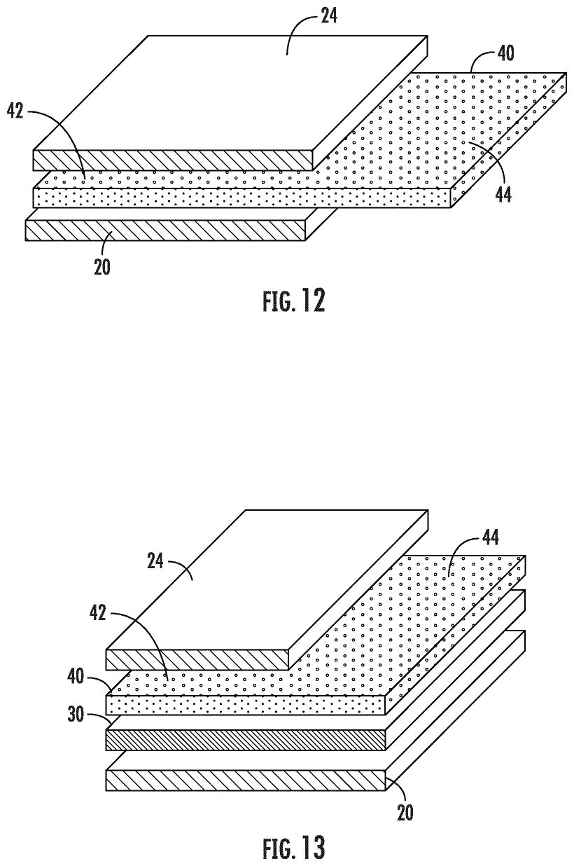

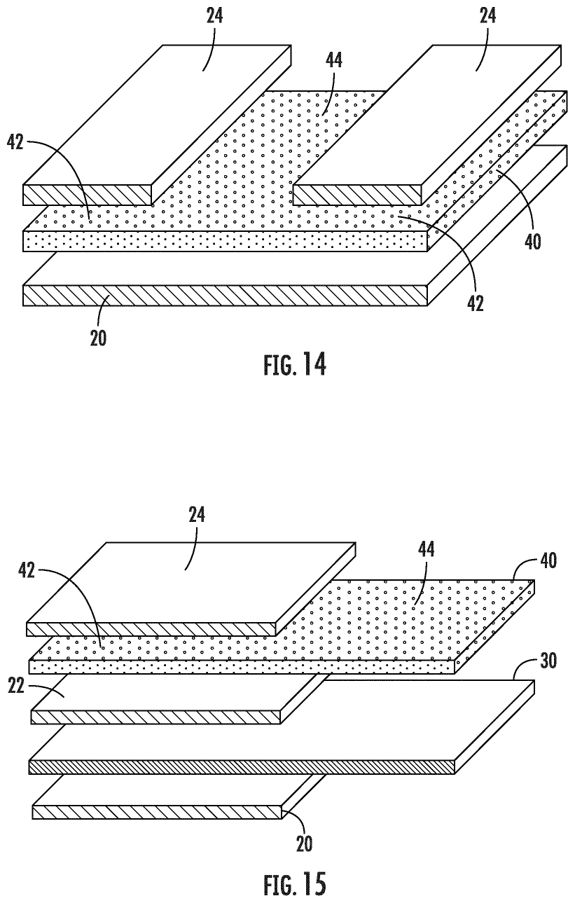

FIGS. 12-15 schematically illustrate various layering configurations for the layers forming the tube 12 according to different embodiments. FIG. 12 illustrates a tube 12 that has no reinforcing element 30 and is formed by an inner film layer 20, a foam material 40, and an outer film layer 24. FIG. 13 illustrates a tube 12 that has the foam material located directly in contact with the outer surface of the reinforcing element 30, i.e., without a middle film layer 22. FIG. 14 illustrates a tube 12 in which the foam material 40 has an exposed portion 44 with covered portions 42 extending axially on both sides of the exposed portion 44. While FIG. 14 illustrates only inner and outer film layers 20, 24, this configuration may be used in an embodiment with a reinforcing element 30, e.g., as in FIG. 7 or FIG. 13. FIG. 15 illustrates an embodiment where both the foam material 40 and the reinforcing element 30 extend beyond the distal end of the film layers 20, 22, 24 at one of the ends 13, 14 of the tube 12, such as in the configuration of FIG. 1. It is understood that the foam material 40 may be provided with a covered portion 42 and an exposed portion 44 that is not covered on the outer surface thereof, but in some embodiments, the entire inner surface of the foam material 40 may be covered by one or more inner layers (e.g., film layers 20, 22).

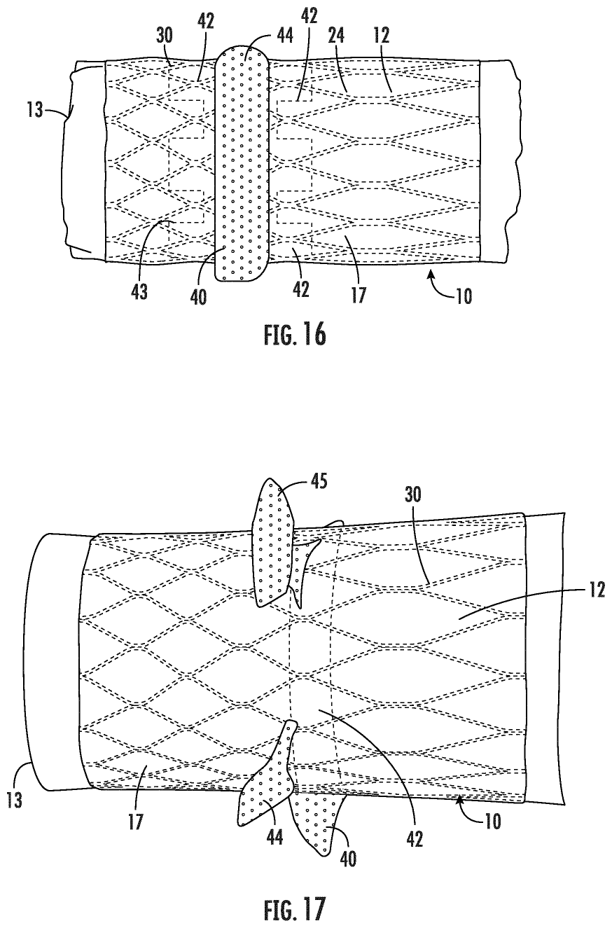

FIGS. 16-26 illustrate additional embodiments of grafts 10 each comprising a tube 12 that includes a structure of layers that is similar to the grafts 10 in FIGS. 1-4 and 5-6, having an inner film layer 20, a reinforcing element 30, a middle film layer 22, a foam material 40, and an outer film layer 24 in layered order. The grafts 10 differ primarily in the position and configuration of the foam material 40.

FIG. 16 illustrates an embodiment of a graft 10 where the foam material 40 is configured as a collar extending around the tube 12 at a location spaced axially inwardly from the ends 13, 14 of the tube 12. The exposed portion 44 of the foam material 40 extends radially outward from the outer surface 17 of the tube, and the covered portion 42 extends axially on both sides of the exposed portion 44. The foam material 40 in this embodiment is formed of a single piece wrapped around the tube 12.

FIG. 17 illustrates an embodiment of a graft 10 where the foam material 40 is configured such that the exposed portion 44 forms a plurality of elongated fins 45 extending outward from the outer surface 17 of the tube 12. The fins 45 are arranged to extend outward around an annular area of the tube 12 at a location spaced inwardly from the ends 13, 14 of the tube 12. In one embodiment, the foam material 40 may be formed as a single piece extending around most or all of the periphery of the tube 12 and having a plurality of fins 45 extending outwardly. In another embodiment, the foam material 40 may be formed as a plurality of discrete portions each having an exposed portion 44 in the form of one or more fins 45 and a covered portion 42 as described herein. In the embodiment of FIG. 17, the covered portion 42 extends axially from one side of the exposed portion 44; however, in another embodiment, covered portions 42 may extend axially from both sides of the exposed portion 44.

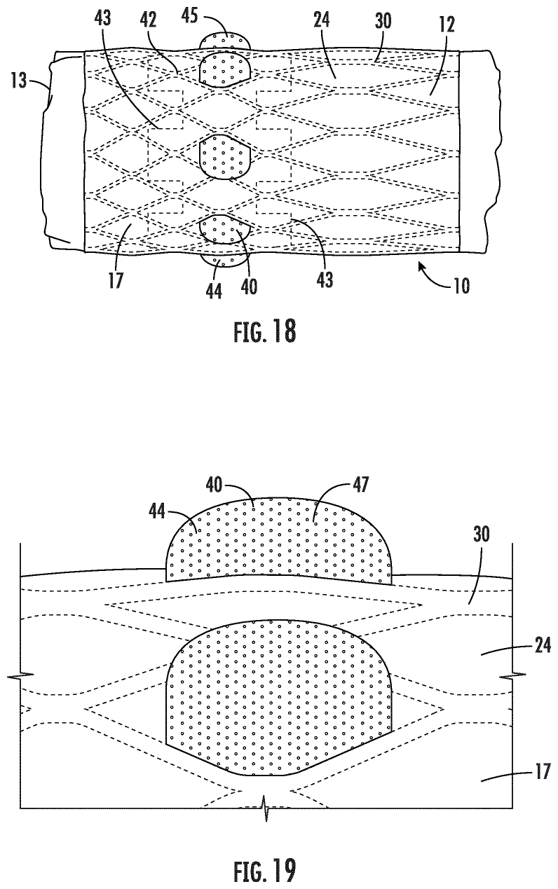

FIGS. 18-19 illustrate an embodiment of a graft 10 where the foam material 40 is configured such that the exposed portion 44 forms a plurality of protrusions 47 extending outward from the outer surface 17 of the tube 12. The protrusions 47 are arranged to extend outward around an annular area of the tube 12 at a location spaced inwardly from the ends 13, 14 of the tube 12. As described herein with respect to FIG. 17, the foam material 40 in this embodiment may be formed as a single piece or a plurality of discrete portions. The graft 10 of FIGS. 18-19 has a covered portion 42 extending axially from both sides of the exposed portion 44.

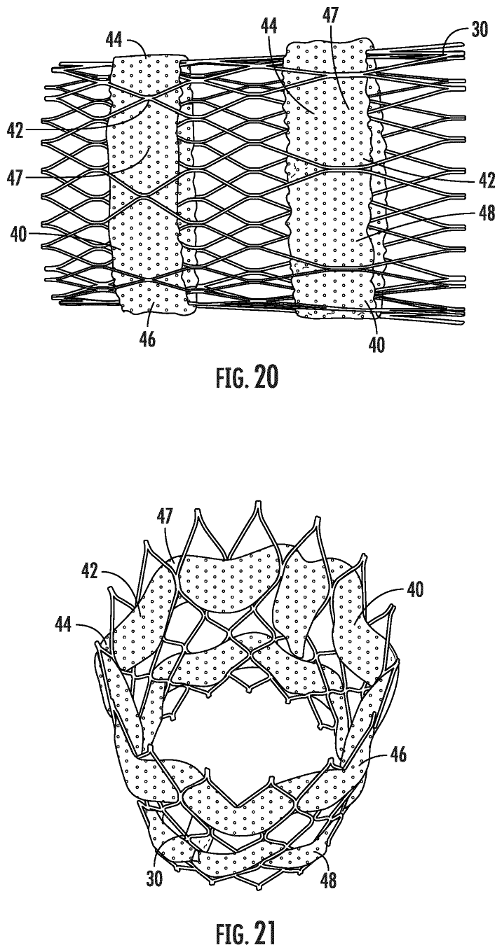

FIGS. 20-21 illustrate an embodiment of a graft 10 where the foam material 40 is intertwined with a reinforcing element 30 in the form of a stent. While the entire tube 12 in this embodiment is not illustrated, the graft 10 may include a layered configuration as described herein, e.g., as shown in FIG. 7. The foam material 40 in this embodiment is configured such that the exposed portion 44 forms a plurality of protrusions 47 extending outward from the outer surface 17 of the tube 12, and the covered portion 42 extends circumferentially between the protrusions 47. The portions of the foam material 40 that extend radially inward of the stent 30 form the covered portion 42, and the portions of the foam material 40 that extend radially outward of the stent 30 form the exposed portion 44. The protrusions 47 are arranged to extend outward around an annular area of the tube 12 at a location spaced inwardly from the ends 13, 14 of the tube 12. In this embodiment, the foam material 40 includes first and second foam members 46, 48 that are spaced axially from each other along the tube 12.

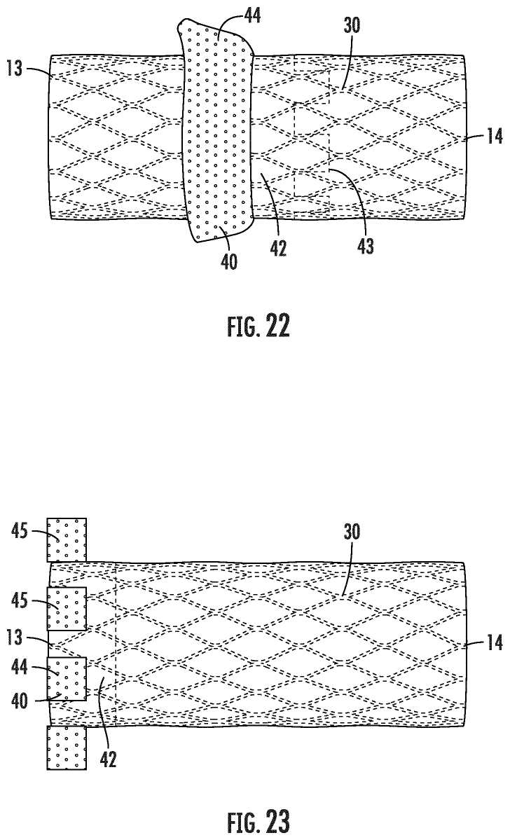

FIG. 22 illustrates an embodiment of a graft 10 where the foam material 40 is configured as a collar extending around the tube 12 at a location spaced axially inwardly from the ends 13, 14 of the tube 12. The exposed portion 44 of the foam material 40 in this embodiment is configured as an annular or frusto-conical collar having a flared configuration that permits the foam material 40 to expand radially at the distal end thereof, similarly to one of the foam members 46, 48 illustrated in FIGS. 5-6. The covered portion 42 of the foam material 40 in this embodiment may be configured similarly to that of FIGS. 5-6 or may extend in both axial directions from the exposed portion 44.

FIG. 23 illustrates an embodiment of a graft 10 where the foam material 40 is configured such that the exposed portion 44 forms a plurality of protrusions 47 extending outward from the outer surface 17 of the tube 12, similar to the embodiment of FIGS. 18-19. The protrusions 47 are arranged to extend outward around an annular area of the tube 12 at one of the ends 13, 14 of the tube 12. The graft 10 of FIGS. 18-19 has a covered portion 42 extending axially inward from the exposed portion 44. Alternately, the configuration shown in FIG. 23 may be formed by the foam material 40 configured similarly to one of the foam members 46, 48 in FIGS. 20-21.

FIG. 24 illustrates an embodiment of a graft 10 where the foam material 40 is configured such that the exposed portion 44 forms a plurality of fins 45 extending outward from the outer surface 17 of the tube 12. The fins 45 are arranged to extend outward around an annular area of the tube 12 at a location spaced inwardly from the ends 13, 14 of the tube 12. The foam material 40 in this embodiment may be configured similarly to the foam material 40 in FIG. 17, with the fins 45 in FIG. 24 having greater width and circumferential extent than the fins 45 in FIG. 17. Similar to FIG. 17, the foam material 40 in this embodiment may be formed as a single piece having a plurality of fins 45 or as a plurality of discrete portions each having an exposed portion 44 in the form of one or more fins 45. The covered portion 42 in this embodiment may extend axially from one side of the exposed portion 44 or from both sides of the exposed portion 44.

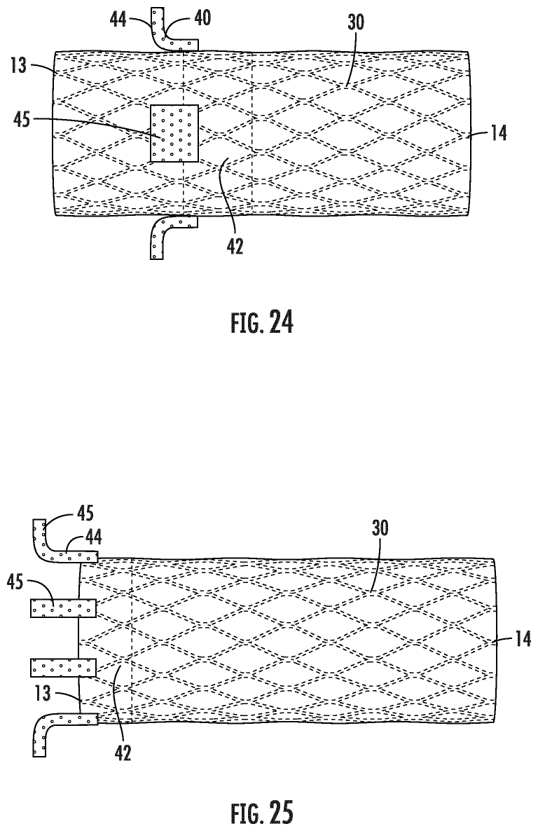

FIG. 25 illustrates an embodiment of a graft 10 where the foam material 40 is configured similarly to the foam material 40 of FIG. 24 (including any variations or alternate embodiments), with the foam material 40 located at one of the ends 13, 14 of the tube 12, rather than between the ends 13, 14. The covered portion 42 in this embodiment extends axially inward from the exposed portion 44.

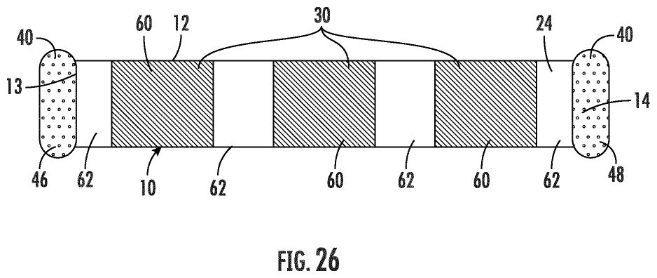

FIG. 26 illustrates an embodiment of a graft 10 where the foam material 40 is configured similarly to the foam material 40 of FIGS. 1-4, and where the tube 12 includes a plurality of separate reinforcing elements 30. The reinforcing elements 30 in this embodiment are in the form of cylindrical stents that are spaced from each other axially along the length of the tube 12. The film layers 20, 22, 24 extend axially between the reinforcing elements 30 and interconnect the reinforcing elements 30. In this embodiment, the tube 12 may be considered to have a plurality of first sections 60 where the graft 10 is reinforced by one of the reinforcing elements 30, and a plurality of second sections 62 extending between the first sections 60. The second sections 62 are formed only of the film layers 20, 22, 24 in this embodiment. Additionally, in one embodiment, the second sections 62 may include corrugations or other structures that function to reinforce the respective second section 62 and/or provide flexibility to the section 62. Further, in one embodiment, the tube 12 may include only a single first section 60, with one or more second sections 62 at one or both ends of the first section 60.

Various sizes and configurations of the first section(s) 60 and second section(s) 60 are possible. Examples of such configurations can be found in U.S. Provisional Patent Application No. 63/300,080, which is incorporated by reference herein in its entirety. The size of a first section 60 is generally dictated by the length of the reinforcing element 30, while the size of the second section 60 is generally dictated by the length of the modification of the film layer(s). Various lengths and sizes of reinforcing elements 30 may be used, such that the graft 10 may have first and second sections 60, 62 of uniform or non-uniform length. The graft 10 may have one or a plurality of the first sections 60 and one or a plurality of the second sections 62, depending on the desired application and properties of the graft 10. In an embodiment, each second section 62 is connected to two first sections 60. In an embodiment, each first section 60 is connected to two second sections 62. In an embodiment, the ends 13, 14 of the tube 12 are second sections 62. In an embodiment, the ends 13, 14 of the tube 12 are first sections 60. It is understood that the foam material 40 may be disposed in the first section(s) 60 and/or the second section(s) 62.

As discussed herein, FIGS. 1-26 illustrate potential embodiments of grafts 10 that include one or more portions of foam material 40, and such embodiments can be combined or modified in various manners. Certain embodiments disclosed herein illustrate a foam material 40 that is configured as a single piece or portion or multiple pieces or portions, and it is understood that any of the embodiments disclosed herein may include any number of pieces or portions of the foam material 40, including multiple similar or identical configurations of the foam material 40 or a mixture of multiple configurations of the foam material 40. For example, in one embodiment, the graft 10 may include foam members having one configuration at the ends 13, 14 of the tube 12 and one or more differently configured foam member located between the ends 13, 14 of the tube 12. It is also understood that multiple different types of foam materials 40 may be used in one embodiment.

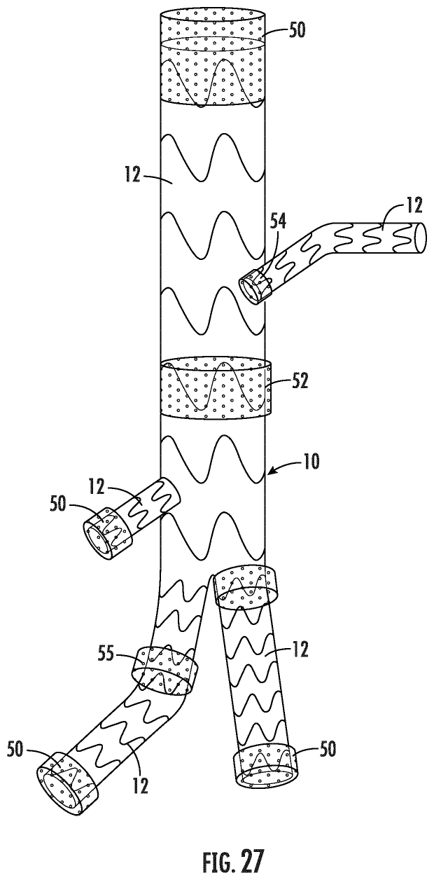

Still further, a graft 10 in some embodiments may include multiple tubes 12 connected together, with a foam material 40 provided in the form of one or more members. As one example, FIG. 27 illustrates an embodiment of a graft 10 that is fenestrated and includes a plurality of tubes 12 configured as a modular or multi-leg graft. The tubes 12 in the graft 10 of FIG. 27 include multiple foam members, including foam members 50 at the distal ends of the graft 10 to resist endoleak and increase anchoring stability, a foam member 52 in the middle of one of the tubes 12 to increase anchoring stability, and foam members 54 at joints between different tubes 12 to increase connection stability at those locations. The foam members 50, 52, 54 may be connected to the tube(s) 12 in various manners described herein, and each foam member 50, 52, 54 may include a covered portion 42 and an exposed portion 44 as described herein. It is understood that FIG. 27 is depicted schematically, and the covered portions 42 and the exposed portions 44 are not illustrated.

As discussed herein, the graft comprises at least two film layers 20, 22, 24, which may be the same or different, and a foam material 40. Other components may be present, such as a reinforcing element 30. Such materials will now be described.

### Films

The tube 12 comprises at least two film layers 20, 22, 24. The film typically forms at least the inner surface 15 of the tube 12. In an embodiment where the tube 12 has multiple sections 60, 62, the film typically extends through each of the one or more first sections 60 and one or more second sections 62 of the tube 12 in a single piece. Thus, in an embodiment, each of the one or more first sections 60 and each of the one or more second sections 62 comprise the film. The film may be formed from multiple pieces, such as by bonding together multiple separate layers of film or by bonding together different sections of film at various interfaces (e.g., by thermal lamination).

One exemplary method of forming the film is to helically wrap the material around a mandrel 19 (see FIG. 4) and bonding the overlapping sections together to form a layer 20, 22, 24 of the film.

The film is typically formed from a polymer material, such as PTFE, such as ePTFE, or UHMWPE (ultra-high molecular weight polyethylene). In an embodiment, the film is porous. In an embodiment, the film is microporous or nanoporous. In an embodiment the film is non-porous.

In one embodiment, the film comprises polyethylene, preferably, ultra-high molecular weight polyethylene (UHMWPE). In an embodiment, the film is a microporous polyethylene film, such as a microporous UHMWPE film, also known as a UHMWPE membrane. A suitable commercially available UHMWPE film is Dyneema Purity® Membrane from DSM. In an embodiment each film layer 20, 22, 24 has a thickness of from 10 to 100 μm, or from 30 to 60 μm. In an embodiment, multiple pieces or sub-layers of microporous UHMWPE film are bonded together using heat (thermal lamination) to create a film layer 20, 22, 24. In such an embodiment, each piece or sub-layer may have a thickness of from 5 to 25 μm, such as about 15 μm. Both porous and non-porous materials may be used.

As described herein, during manufacturing, the film layers 20, 22, 24 and the foam material 40 are heated to a lamination temperature sufficient to soften the film layers 20, 22, 24 to permit lamination, but below the degradation temperature of the foam material 40 or another temperature that significantly changes an important property of the foam material 40 (e.g., structural changes). In an embodiment, the lamination temperature of the material(s) of the film layers 20, 22, 24 is in a range having a lower end of 100° C., 110° C., 120° C., 130° C., or 140° C., and an upper end of 140° C., 150° C., 160° C., 170° C., 180° C., 190° C., or 200° C. The material may also have a melting point within these ranges. Such a low lamination temperature allows for processing at a temperature that is less than the degradation temperature of the foam. For example, the film may have a lamination temperature of 130-160° C. in one embodiment, or 135-145° C. in another embodiment.

### Reinforcing Element

In an embodiment, the graft 10 comprises a reinforcing element 30. In an embodiment, the reinforcing element 30 is a stent. Various designs of stents may be suitable. In an embodiment, the reinforcing element 30 is a mesh stent. In an embodiment, the reinforcing element 30 is a zigzag stent. A zigzag stent may comprise multiple cylindrical elements in the form of crowns. The cylindrical elements are connected by bridges. In an embodiment, the reinforcing element 30 is a helical wire. In an embodiment, the stent comprises a plurality of cylindrical elements, such as a plurality of bands or crowns of a zigzag stent. In an embodiment, the stent comprises a plurality of cylindrical elements connected by one or more bridges. Typical stent materials are generally suitable for the reinforcing element 30.

### Foam

The tube 12 comprises a foam material 40 forming at least one foam member. In an embodiment, the foam material 40 is an open-cell foam. The foam material 40 is biocompatible. In an embodiment, the foam material 40 is biostable. In an embodiment, the foam material 40 is biodegradable. In an embodiment, the foam is elastomeric. In an embodiment, the foam material 40 comprises a polyurethane. In an embodiment, the foam material 40 comprises a thermoset polyurethane.

In an embodiment, the foam material 40 has a continuous and interconnected void phase. A continuous and interconnected void phase is a continuous network of structure defining a void space therein, wherein said void space comprises a plurality of interconnected pores forming a continuous network of intercommunicating passageways. In an embodiment, the continuous and interconnected void phase extends from a surface of the foam. In an embodiment, the continuous and interconnected void phase extends through the foam material 40 such that the foam material 40 has fluid permeability from one surface to another. In an embodiment the foam material 40 comprises an internal continuous and interconnected void phase and an external skin layer. In an embodiment, the continuous and interconnected void phase is formed by reticulation as hereinafter described.

In an embodiment, the foam material 40 is coated with a material to encourage cellular ingrowth or proliferation. In an embodiment, such coating comprises collagen, fibronectin, elastin, hyaluronic acid, or a mixture thereof. In an embodiment, a thin layer of collagen is coated on a surface of the foam material 40 to create a boundary layer on the surface. In an embodiment, the foam material 40 is coated with a bioactive agent or a coating comprising a bioactive agent.

In an embodiment, foam material as described in U.S. Pat. No. 7,803,395 or U.S. Pat. No. 9,050,176, which are each hereby incorporated by reference in their entirety, may be used as a foam material 40 for the graft 10 as disclosed herein.

In an embodiment, the foam material 40 has a plurality of interconnected pores with an average diameter or other largest transverse dimension (average cell size) of at least about 50 μm. In an embodiment, the void space comprises from about 70% to about 99% of the volume of the foam material 40 in its uncompressed state. In an embodiment, the average cell size is at least 50, 75, 100, 150, 200, 300, 350, or 400 μm. In an embodiment, the average cell size is at most 1000, 950, 900, 850, 800, 750, 700, 650, 600, 550, 500, 450, or 400 μm.

In an embodiment, the foam material 40 is compressed from its neutral state in a plurality of locations, and the foam material 40 comprises at least one uncompressed location as well. For example, in one embodiment, the foam material 40 may be compressed in the covered portions 42 and uncompressed in the exposed portions 44. In the compressed locations, the foam material 40 may be at least radially compressed, and may be compressed in other directions as well. In an embodiment, the foam material 40 has a density ratio from its most compressed location to its least compressed location of from 15:1, 12:1, 10:1 or 9:1 to 2:1, 3:1, 4:1, 5:1, or 6:1. In an embodiment, the foam material 40 has a density ratio from its most compressed location to least compressed location of about 8:1. In other embodiments, the foam material 40 may be compressed in only some of the covered portions 42, or the foam material 40 may not be compressed in this manner. As another example, the foam material 40 may additionally or alternately be compressed at some or all of the exposed portions 44. The exposed portions 44 of the foam material 40 can be compressed by physically compressing the foam material 40 and then heating the foam material 40 to an appropriate temperature, e.g., 120° C., in an embodiment where a thermoset foam material 40 is used. Such physical compression can be accomplished in one example by wrapping the exposed portions 44, e.g., using a release layer (ePTFE/PTFE tape or similar). The release layer can be removed from the exposed portions 44 after heating, as desired. In this embodiment, the compression of the exposed portions 44 of the foam material 40 could be set during thermal bonding of the film layers 20, 22, 24. Compressing the exposed portions 44 of the foam material 44 in this manner would allow modification and/or control of the size and mechanical properties of the foam material 40 at the exposed portions 44.

In an embodiment, the bulk density of the foam material 40, prior to any compression, may be from about 0.008 g/cc to about 0.96 g/cc. In another embodiment, the bulk density is at least 0.008, 0.010, 0.015, 0.020, 0.025, or 0.03 g/cc. In an embodiment, the bulk density is at most 0.96, 0.75, 0.50, 0.40, 0.30, 0.288, 0.25, 0.20, 0.15, 0.12, 0.115, or 0.104 g/cc. In another embodiment, the bulk density may be from about 0.016 g/cc to about 0.56 g/cc. In another embodiment, the bulk density may be from about 0.008 g/cc to about 0.15 g/cc. In another embodiment, the bulk density may be from about 0.008 g/cc to about 0.127 g/cc. In another embodiment, the bulk density may be from about 0.008 g/cc to about 0.288 g/cc. In another embodiment, the bulk density may be from about 0.016 g/cc to about 0.115 g/cc. In another embodiment, the bulk density may be from about 0.024 g/cc to about 0.104 g/cc. Bulk density is as measured pursuant to the test method described in ASTM Standard D3574.

In an embodiment, the foam material 40 has a compressive strength of at least 3, 4, 5, 10, 15, or 20 kPa. In an embodiment, the foam material 40 has a compressive strength of at most 70, 60, 50, 40, 30, 20, 15, 14, 13, 12, 11, or 10 kPa. In an embodiment, the foam material 40 has a tensile strength in both the parallel and perpendicular directions of at least 10, 11, 12, 13, 14, 15, 16, 17, 18, 19, 20, 21, 22, 23, 24, or 25 kPa. In an embodiment, the foam material 40 has an elongation at break in both the parallel and perpendicular directions of at least 100, 110, 120, 130, 140, 150, 16, 170, or 180%.

The foam material 40 may exhibit resilience, that is it is able to recoil or spring back into shape after bending, stretching, or being compressed. Such feature may allow for targeted delivery and release at a surgical delivery using minimally invasive means, such as by, e.g., catheter, endoscope, arthroscope, laparoscope, cystoscope or syringe. Upon delivery at the target site, the foam material 40 may substantially regain its shape.

The foam material 40 is typically formed from a matrix. In an embodiment, the matrix is a polymer. In an embodiment, the matrix is formed by a reaction of a mixture comprising: (i) a polyol, and (ii) an isocyanate component. In an embodiment, the mixture further comprises a chain extender. In an embodiment, the matrix is thermoset. In an embodiment, the matrix is thermoplastic. In an embodiment, elastomeric matrix is free of allophanate and biuret linkages.

An elastomeric matrix comprising a continuous network of intercommunicating passageways may be produced using a process comprising: (a) synthesizing a polycarbonate polyurethane foam comprising a plurality of cell walls defining a plurality of pores therein by reacting a mixture comprising: (i) a polycarbonate polyol, (ii) an isocyanate component, and (iii) a blowing agent for forming said plurality of pores; and (b) igniting a combustible gas to remove at least about 40% of said plurality of cell walls to form said continuous network of intercommunicating passageways.

In an embodiment, the foam material 40 comprises a polyurethane. The polyurethane comprises the reaction product of an isocyanate and a polyol. In an embodiment, the polyurethane comprises the reaction product of a diisocyanate, a polymeric aliphatic diol, and optionally a chain extender. In an embodiment, the polyurethane consists of the reaction product of a diisocyanate, a polymeric aliphatic diol, and a chain extender diol. In an embodiment, the polyurethane is linear.

By a reaction product, it is meant that the isocyanate and polyol are engaged in a simultaneous or sequential chemical reaction. For example, a reaction product of a diisocyanate, a polymeric aliphatic diol, and a chain extender diol is formed i) when the diisocyanate, polymeric aliphatic diol, and chain extender diol are all reacted together in a single solution, or ii) when a prepolymer is first formed by reacting the diisocyanate and the polymeric aliphatic diol, and then this prepolymer is subsequently reacted with a chain extender diol.

The components of an exemplary polyurethane comprising a diisocyanate, a polymeric aliphatic diol, and a chain extender will now be described.

### Diisocyanate

The polyurethane comprises the residue of a diisocyanate. In an embodiment, the diisocyanate is aliphatic. In an embodiment, the diisocyanate is aromatic. In an embodiment, the diisocyanate comprises 2,4′-diphenylmethane diisocyanate, 4,4′-diphenylmethane diisocyanate (MDI), 2,4-toluene diisocyanate, 2,6-toluene diisocyanate, 1,4-phenylene diisocyanate, hexamethylene diisocyanate (HDI), tetramethylene-1,4-diisocyanate, cyclohexane-1,4-diisocyanate, dicyclohexylmethane-4,4′-diisocyanate (HMDI), isophorone diisocyanate (IPDI), or a mixture thereof. In an embodiment, the diisocyanate comprises hexamethylene diisocyanate, dicyclohexylmethane 4,4′-diisocyanate, isophorone diisocyanate, or a mixture thereof. In an embodiment, the diisocyanate consists of hexamethylene diisocyanate, dicyclohexylmethane 4,4′-diisocyanate, isophorone diisocyanate, or a mixture thereof. In an embodiment, the diisocyanate comprises 4,4′-diphenylmethane diisocyanate (MDI), 2,4-toluene diisocyanate, 2,6-toluene diisocyanate, or 1,4-phenylene diisocyanate. In an embodiment, the diisocyanate consists of 4,4′-diphenylmethane diisocyanate (MDI), 2,4-toluene diisocyanate, 2,6-toluene diisocyanate, 1,4-phenylene diisocyanate, or a mixture thereof. In an embodiment, the diisocyanate comprises 2,4′-diphenylmethane diisocyanate, 4,4′-diphenylmethane diisocyanate, or a mixture thereof.

In an embodiment, the molecular weight of the diisocyanate is from 100 to 500 g/mol. In an embodiment, the molecular weight of the diisocyanate is from 150 to 260 g/mol.

In an embodiment, the formulation from which the polyurethane is formed comprises at least 10 wt %, at least 20 wt %, at least 25 wt %, at least 30 wt %, at least 35 wt %, or at least 40 wt % of a diisocyanate, based on the total weight of the formulation. In an embodiment, the formulation from which the polyurethane is formed comprises at most 50 wt %, at most 40 wt %, at most 35 wt %, at most 30 wt %, at most 25 wt %, or at most 20 wt % of a diisocyanate, based on the total weight of the formulation. In an embodiment, the polyurethane comprises at least 10 wt %, at least 20 wt %, at least 25 wt %, at least 30 wt %, at least 35 wt %, or at least 40 wt % of the residue of a diisocyanate, based on the polyurethane. In an embodiment, the polyurethane comprises at most 50 wt %, at most 40 wt %, at most 35 wt %, at most 30 wt %, at most 25 wt %, or at most 20 wt % of the residue of a diisocyanate, based on the total weight of the polyurethane.

### Polymeric Polyol

The polyurethane comprises the residue of a polymeric polyol. In an embodiment, the polyurethane comprises the residue of a polymeric diol. A polymeric polyol comprises at least two OH groups and a backbone. The OH groups may be directly attached to the backbone or may be separated by a linker. For example, a hydroxyalkyl terminated polydimethylsiloxane (carbinol terminated) is a polymeric diol.

In an embodiment, the polymeric polyol comprises an aliphatic polymeric polyol. In an embodiment, the polymeric polyol comprises an aromatic polymeric polyol.

In an embodiment, the polymeric polyol comprises a poly(alkylene oxide), a polycarbonate, a polysiloxane, a random or block copolymer thereof, or a mixture thereof. In an embodiment, the polymeric polyol comprises a poly(alkylene oxide), a polycarbonate, a random or block copolymer thereof, or a mixture thereof. In an embodiment, the polymeric polyol comprises C₂-C₁₆ fluoroalkyl or C₂-C₁₆ fluoroalkyl ether.

In an embodiment, a difunctional polymeric polyol cannot, on its own, induce sufficient crosslinking for the polyurethane foam. Therefore, a higher functionality polyol, such as a triol or tetraol is used.

In an embodiment, the polymeric polyol comprises a poly(ethylene oxide) diol, a poly(propylene oxide) diol, a poly(tetramethylene oxide) diol, a poly(isobutylene) diol, a polyester diol, for example a polyester diol formed from adipic acid or isophtalic acid and a monomeric diol, an alkane diol, such as a hydrogenated polybutadiene diol or a polyethylene diol, a poly(hexamethylene carbonate) diol, a poly(polytetrahydrofuran carbonate) diol, a polysiloxane diol, a random or block copolymer diol of poly(ethylene oxide) and poly(propylene oxide), a random or block copolymer diol of poly(ethylene oxide) and poly(tetramethylene oxide), a random or block copolymer diol of poly(ethylene oxide) and a polysiloxane, or a mixture thereof.

In an embodiment, the polymeric polyol comprises a polycarbonate diol. In an embodiment, the polymeric aliphatic diol comprises a polycarbonate diol that comprises a poly(hexamethylene carbonate) diol or a poly(polytetrahydrofuran carbonate) diol. In an embodiment, the polymeric diol comprises a polycarbonate diol having a Mn of at least 500 g/mol, at least 750 g/mol, at least 1000 g/mol, or at least 1500 g/mol. In an embodiment, the polymeric aliphatic diol comprises a polycarbonate diol having a Mn of at most 10,000 g/mol, at most 7500 g/mol, at most 5000 g/mol, at most 4000 g/mol, at most 3000 g/mol, or at most 2500 g/mol.

In an embodiment, the polymeric polyol comprises a polysiloxane diol, a polycarbonate diol, or a poly(tetramethylene oxide) diol. In an embodiment, the polymeric aliphatic diol consists of a polysiloxane diol, a polycarbonate diol, a poly(tetramethylene oxide) diol, or a mixture thereof. In an embodiment, the polymeric diol comprises a mixture of two or more of a polysiloxane diol, a polycarbonate diol, or a poly(tetramethylene oxide) diol. In an embodiment, the polymeric diol consists of a mixture of two or more of a polysiloxane diol, a polycarbonate diol, or a poly(tetramethylene oxide) diol.

In an embodiment, the polymeric polyol has a Mn of at least 200 g/mol, at least 250 g/mol, at least 300 g/mol, at least 400 g/mol, or at least 500 g/mol, at least 600 g/mol, at least 700 g/mol, at least 800 g/mol, at least 900 g/mol, or at least 1000 g/mol. In an embodiment, the polymeric aliphatic diol has a Mn of at most 10,000 g/mol, at most 8500 g/mol, at most 6000 g/mol, at most 5000 g/mol, at most 4000 g/mol, at most 3000 g/mol, at most 2000 g/mol, or at most 1500 g/mol.

In an embodiment, the polyurethane is formed from a formulation that comprises at least 20 wt %, at least 30 wt %, at least 40 wt %, at least 50 wt %, or at least 60 wt % of a polymeric aliphatic polyol, based on the total weight of the formulation. In an embodiment, the polyurethane is formed from a formulation that comprises at most 80 wt %, at most 70 wt %, at most 60 wt %, or at most 50 wt % of a polymeric polyol, based on the total weight of the formulation. In an embodiment, the polyurethane comprises at least 20 wt %, at least 30 wt %, at least 40 wt %, at least 50 wt %, or at least 60 wt % of the residue of a polymeric aliphatic polyol, based on the total weight of the polyurethane. In an embodiment, the polyurethane comprises at most 80 wt %, at most 70 wt %, at most 60 wt %, or at most 50 wt % of the residue of a polymeric polyol, based on the total weight of the polyurethane.

### Chain Extender

In an embodiment, the polyurethane comprises the residue of a chain extender. The chain extender is a low molecular weight polyol, typically a diol. A triol or higher functional chain extender may be used if cross-linking is desired. In an embodiment, the chain extender is a diol and the polyurethane is a thermoplastic. In an embodiment, the chain extender is a diol and the polyurethane is a thermoset. In an embodiment, the chain extender is a diol and the polyurethane is a thermoplastic.

In an embodiment, the polyurethane comprises the residue of a chain extender diol. A chain extender diol is a non-polymeric diol having a molecular weight of 500 g/mol or less. In an embodiment, the chain extender diol is an alkane diol having from 2 to 20 carbon atoms, wherein one or more carbon atoms may be substituted with oxygen, silicon, phosphorous, or sulfur. In an embodiment, the chain extender diol is an alkane diol having from 2 to 20 carbon atoms, wherein one or more carbon atoms may be substituted with oxygen or silicon. In an embodiment, the chain extender diol is an alkane diol having from 2 to 20 carbon atoms, wherein one or more carbon atoms may be substituted with oxygen. In an embodiment, the chain extender diol is an unsubstituted alkane diol having from 2 to 20 carbon atoms.

An unsubstituted alkane diol is a diol consisting of single-bonded carbon and hydrogen atoms and two OH groups. A substituted alkane diol would be an alkane diol but for the substitution of one or more carbon atoms with another atom, such as oxygen or silicon, while still retaining at least two carbon atoms. Examples of unsubstituted alkane diols are ethylene glycol, propanediol, butanediol, pentanediol, 1,4-cyclohexanedimethanol, and the like. Examples of substituted alkane diols are diethylene glycol, dipropylene glycol, 1,3-bis(4-hydroxybutyl)tetramethyldisiloxane (BHTD), 1,3-bis(hydroxypropyl)tetramethyldisiloxane, 1,3-bis(3-hydroxyisobutyl)tetramethyldisiloxane, 3-ethoxy-1,2-propanediol, or 2,2′-Thiodiethanol.

In an embodiment, the chain extender diol comprises ethylene glycol, diethylene glycol, propylene glycol, dipropylene glycol, 1,3-propanediol, 1,2-butanediol, 1,3-butanediol, 1,4-butanediol, 2,3-butanediol, 1,2-pentanediol, 1,3-pentanediol, 1,4-pentanediol, 1,5-pentanediol, 1,3-hexanediol, 1,4-hexanediol, 1,5-hexanediol, 1,6-hexanediol, 2,4-hexanediol, 2,5-hexanediol, 1,2-octanediol, 1,3-octanediol, 1,4-octanediol, 1,5-octanediol, 1,6-octanediol, 1,7-octanediol, 1,8-octanediol, 2,7-octanediol, neopentyl glycol, 1,2-cyclohexanediol, 1,3-cyclohexanediol, 1,4-cyclohexanediol, 1,2-cyclohexanedimethanol, 1,3-cyclohexanedimethanol, 1,4-cyclohexanedimethanol, or 1,1-cyclohexanedimethanol. In an embodiment, the chain extender diol comprises ethylene glycol, propylene glycol, 1,3-propanediol, 1,2-butanediol, 1,3-butanediol, 1,4-butanediol, 2,3-butanediol, 1,2-pentanediol, 1,3-pentanediol, 1,4-pentanediol, 1,5-pentanediol, 1,3-hexanediol, 1,4-hexanediol, 1,5-hexanediol, 1,6-hexanediol, 2,4-hexanediol, 2,5-hexanediol, 1,2-octanediol, 1,3-octanediol, 1,4-octanediol, 1,5-octanediol, 1,6-octanediol, 1,7-octanediol, 1,8-octanediol, or 2,7-octanediol.

In an embodiment, the chain extender has a molecular weight of at least 60 g/mol, at least 70 g/mol, at least 80 g/mol, at least 90 g/mol, or at least 100 g/mol. In an embodiment, the chain extender has a molecular weight of at most 500 g/mol, at most from 400 g/mol, at most 300 g/mol, at most 200 g/mol, or at most 150 g/mol.

In an embodiment, the chain extender comprises a polyol having a functionality of at least 3. In embodiment, the chain extender comprises a monomeric triol or tetraol, or a propoxylate thereof. In an embodiment, the chain extender comprises glycerol, glycerol propoxylate, glycerol ethoxylate, 1,2,4-benzenetriol, 3-methyl-1,3,5-pentanetriol, pentaerythritol, pentaerythritol propoxylate, or pentaerythritol ethoxylate. In an embodiment, the chain extender has a molecular weight of from 90 to 500 g/mol. In an embodiment, the chain extender has a molecular weight of from 90 to 280 g/mol.

In an embodiment, the polyurethane is formed from a formulation that comprises at least 1 wt %, at least 2 wt %, at least 5 wt %, at least 8 wt %, or at least 10 wt % of a chain extender diol, based on the total weight of the formulation. In an embodiment, the polyurethane is formed from a formulation that comprises at most 20 wt %, at most 15 wt %, at most 12 wt %, at most 10 wt %, at most 8 wt %, or at most 5 wt %, of a chain extender diol, based on the total weight of the formulation. In an embodiment, the polyurethane comprises at least 1 wt %, at least 2 wt %, at least 5 wt %, at least 8 wt %, or at least 10 wt % of the residue of a chain extender diol, based on the total weight of the polyurethane. In an embodiment, the polyurethane comprises at most 20 wt %, at most 15 wt %, at most 12 wt %, at most 10 wt %, at most 8 wt %, or at most 5 wt %, of the residue of a chain extender diol, based on the total weight of the polyurethane.

### Endgroups

In an embodiment, the polyurethane comprises one or more endgroups. An endgroup is a moiety present at a terminal end of a molecule. In an embodiment, the polyurethane is linear and comprises an endgroup at each terminus of the backbone. In an embodiment, the endgroup is linear. In an embodiment, the endgroup is branched. In an embodiment, the polyurethane comprises an average of at least 0.1 endgroups, at least 0.25 endgroups, at least 0.5 endgroups, at least 1 endgroup, at least 1.5 endgroups, at least 1.8 endgroups, about 2 endgroups, or at least 2 endgroups. In an embodiment, the polyurethane comprises an average of at most 4 endgroups an average of at most 2 endgroups, or an average of at most 2 endgroups.

An endgroup may be formed by reacting a terminal isocyanate group present after forming the polymer backbone with a coreactive group on a monofunctional moiety. For instance, a terminal isocyanate group may be reacted with 1-octanol or octylamine to form a C₈ alkyl endgroup. Endgroups may also result from the inclusion of chain stoppers, such as monofunctional alcohols, in a formulation used in the formation of a polyurethane. For instance, a formulation for forming a polyurethane may comprise a diisocyanate, a polymeric aliphatic diol, a chain extender, and a monofunctional alcohol.

In an embodiment, the endgroup comprises a hydrophobic poly(alkylene oxide), a hydrophilic poly(alkylene oxide), a copolymer comprising a hydrophilic poly(alkylene oxide) and a hydrophobic poly(alkylene oxide), a polysiloxane, C₂-C₂₀ alkyl, C₂-C₁₆ fluoroalkyl, C₂-C₁₆ fluoroalkyl ether, or copolymers thereof. In an embodiment, the polysiloxane is a poly(dimethylsiloxane). In an embodiment, the hydrophilic poly(alkylene oxide) is poly(ethylene oxide). In an embodiment, the hydrophobic poly(alkylene oxide) is poly(propylene oxide) or poly(tetramethylene oxide). In an embodiment, the endgroup comprises a hydrophobic poly(alkylene oxide), a hydrophilic poly(alkylene oxide), a copolymer comprising a hydrophilic poly(alkylene oxide) and a hydrophobic poly(alkylene oxide), C₂-C₂₀ alkyl, C₂-C₁₆ fluoroalkyl, C₂-C₁₆ fluoroalkyl ether, or copolymers thereof. Such endgroups may be formed with monofunctional alcohols, including carbinols, or amines of the foregoing.

In an embodiment, the endgroup comprises C₂-C₁₆ fluoroalkyl or C₂-C₁₆ fluoroalkyl ether.

Such endgroups may be formed with monofunctional alcohols or amines comprising C₂-C₁₆ fluoroalkyl or C₂-C₁₆ fluoroalkyl ether.

In an embodiment, the endgroup is formed from a monofunctional alcohol or amine comprising C₂-C₁₆ fluoroalkyl or C₂-C₁₆ fluoroalkyl ether. In an embodiment, the endgroup is formed from 1H, 1H-Perfluoro-3,6-dioxaheptan-1-ol, 1H, 1H-Nonafluoro-1-pentanol, 1H, 1H-Perfluoro-1-hexyl alcohol, 1H, 1H-Perfluoro-3,6,9-trioxadecan-1-ol, 1H, 1H-Perfluoro-1-heptyl alcohol, 1H, 1H-Perfluoro-3,6-dioxadecan-1-ol, 1H, 1H-Perfluoro-1-octyl alcohol, 1H, 1H-Perfluoro-1-nonyl alcohol, 1H, 1H-Perfluoro-3,6,9-trioxatridecan-1-ol, 1H, 1H-Perfluoro-1-decyl alcohol, 1H, 1H-Perfluoro-1-undecyl alcohol, 1H, 1H-Perfluoro-1-lauryl alcohol, 1H, 1H-Perfluoro-1-myristyl alcohol, or 1H, 1H-Perfluoro-1-palmityl alcohol.

In an embodiment, the endgroup is monomeric and has a molecular weight of 200 g/mol or more, 300 g/mol or more, or 500 g/mol or more. In an embodiment, the endgroup is monomeric and has a molecular weight of 1,000 g/mol or less or 800 g/mol or less. In an embodiment, the endgroup is polymeric and has a Mn of 10,000 g/mol or less, 8,000 g/mol or less, 6,000 g/mol or less, or 4,000 g/mol or less. In an embodiment, the endgroup is polymeric and has a Mn of 500g/mol or more, 1,000 g/mol or more, or 2,000 g/mol or more.

In an embodiment, the endgroup is present in an amount of at least 0.1 wt %, at least 0.2 wt %, at least 0.3 wt %, or at least 0.5 wt %, based on the total weight of the formulation from which the polyurethane is formed. In an embodiment, the endgroup is present in an amount of at most 3 wt %, at most 2 wt % or at most 1 wt %, based on the total weight of the formulation from which the polyurethane is formed. In an embodiment, the endgroup is present in an amount of at least 0.1 wt %, at least 0.2 wt %, at least 0.3 wt %, or at least 0.5 wt %, based on the total weight of the polyurethane. In an embodiment, the endgroup is present in an amount of at most 3 wt %, at most 2 wt % or at most 1 wt %, based on the total weight of the polyurethane.

### Formation of Polyurethanes

The polyurethanes may be formed as generally known in the art. A catalyst may be employed. In an embodiment, the catalyst is stannous octoate or dibutyltin dilaurate. Amine catalysts may also be used.

In an embodiment, the polyurethane is cross-linked. In an embodiment, the foam comprises a cross-linked polyurethane. Certain polyurethanes may require cross-linking to achieve a stable foam such that the foam does not collapse. In order to cross-link a polyurethane, a 3+ functional compound, such as a tri-isocyanate, and/or a small quantity of an optional ingredient, such as a 3+ functional hydroxyl compound or other crosslinker having a functionality greater than

### Foam Formation

Foam materials 40 can be made using various procedures known in the art. Exemplary procedures for forming a foam material 40 are described in the following paragraphs.

In an embodiment, a prepolymer is first prepared by a conventional method from at least one isocyanate component (e.g., MDI) and at least one multi-functional soft segment material with a functionality greater than 2 (e.g., a polyether-based soft segment with a functionality of 3). Then, the prepolymer, optionally with a catalyst and at least one difunctional chain extender (e.g., 1,4-butanediol) are admixed in a mixing vessel to cure or crosslink the mixture. In another embodiment, crosslinking and foaming, i.e., pore formation, take place together. In another embodiment, crosslinking and foaming take place together in a mold.

In an embodiment, the polyol component is admixed with the isocyanate component and cell opener to form a first liquid. Other optional additives, such as a viscosity modifier, surfactant, chain extender and cross linker are admixed to form a catalyst batch mixture. Then, the first liquid, and the catalyst batch mixture are admixed in a mixing vessel to be foamed and cross-linked. In another embodiment, foaming and cross-linking occur simultaneously. In another embodiment, this foaming mix is poured optionally through a nozzle into a mold and allowed to rise.

Alternatively, a so-called “one-shot” approach may be used. A one-shot embodiment requires no separate prepolymer-making step. In an embodiment, the materials are admixed in a mixing vessel and then foamed and crosslinked.

In another embodiment, all of the ingredients except for the isocyanate component are admixed in a mixing vessel. The isocyanate component is then added, e.g., with high-speed stirring, and crosslinking and foaming ensue. In another embodiment, this foaming mix is poured into a mold and allowed to rise.

Another embodiment involves admixing a polyol component with an isocyanate component and other optional additives, such as a viscosity modifier, surfactant and/or cell opener, to form a first liquid. Next, a second liquid is formed by admixing a blowing agent and optional additives, such as gelling catalyst and/or blowing catalyst. Then, the first liquid and the second liquid are admixed in a mixing vessel and then foamed and crosslinked. The foaming mix is poured optionally through a nozzle into a mold and allowed to rise.

In another embodiment of the one-shot approach, the isocyanate component forms a first liquid. In one embodiment, the isocyanate component is maintained between 5 psi and 30 psi above the ambient pressure and in another embodiment, the isocyanate component is optionally maintained between 20° C. to 30° C. The polyol component is admixed with other optional additives, such as a viscosity modifier, and/or cell opener, to form a second liquid. In an embodiment, the polyol component is admixed or pre-mixed with cell opener and viscosity depressant. In another the polyol component is optionally admixed or pre-mixed with cell opener and viscosity depressant. Next, a third liquid is formed by admixing a blowing agent and a cross-liner and optionally a chain extender and optional additives, such as gelling catalyst and/or blowing catalyst and surfactants. The blowing agent is preferably water and in embodiment is distilled water. The cross-linking agent is glycerol. In one embodiment the blowing agent, water, and cross-linking agent, glycerol, are always admixed before the foaming and cross-linking reactions. Then, the first liquid, the second liquid and the third liquid are admixed in a mixing vessel and then foamed and cross-linked.

Considerations should be taken to ensure that the foaming fluid or the reacting mix is laid down on to the mold bottom surface in a linear fashion or without effective retracing of the flow paths so that it does not introduce any flow disturbances or mix up of the differently aged foaming fluid or the reacting mix coming out of the mixing vessel. In one embodiment, the foaming fluid or the reacting mix is laid down on to the mold bottom surface in a linear fashion or without effective retracing of the flow paths such that the foaming fluid or the reacting mix coming out of the mixing vessel at a later time do not introduce any flow disturbances or mix with foaming fluid or the reacting mix that came out earlier.

At the end of the foam rise, the foaming and cross-linking reaction are considered to be complete or substantially complete, thereby resulting in a matrix in the form of a foamed block or shape. In one embodiment, the matrix is then optionally subjected to additional curing at an elevated temperature. The curing ensures the utilization and/or removal of any free isocyanates and amines and/or completion or substantial completion of other un-reacted ingredients that may not have reacted during foam formation. The curing temperature can range from 70° C. to 120° C. and in other embodiments can range from 75° C. to 110° C. The curing time can range from 30 minutes to 400 minutes and in other embodiment can range from 60 minutes to 300 minutes. In one embodiment, the foamed matrix is not subjected to additional curing at an elevated temperature.

In an embodiment, the continuous and interconnected void phase is formed by reticulation. Reticulation generally refers to a process for at least partially removing cell walls, not merely rupturing or tearing them by a crushing process, which crushing process may undesirably create debris that must be removed by further processing. In another embodiment, the reticulation process substantially fully removes at least a portion of the cell walls. Reticulation may be effected, for example, by at least partially dissolving away cell walls, known variously as “solvent reticulation” or “chemical reticulation”; or by at least partially melting, burning and/or exploding out cell walls, known variously as “combustion reticulation”, “thermal reticulation” or “percussive reticulation”. In an embodiment, two reticulation steps are used, such as a first combustion reticulation followed by a second combustion reticulation. In another embodiment, combustion reticulation is followed by chemical reticulation. In another embodiment, chemical reticulation is followed by combustion reticulation.

One embodiment employs chemical reticulation, where the matrix is reticulated in an acid bath comprising an inorganic acid. Another embodiment employs chemical reticulation, where the matrix is reticulated in a caustic bath comprising an inorganic base. Another embodiment employs solvent reticulation, where a volatile solvent that leaves no residue is used in the process. Another embodiment employs solvent reticulation at a temperature elevated above 25° C. In another embodiment, a matrix comprising a polycarbonate polyurethane is solvent reticulated with a solvent selected from tetrahydrofuran (“THF”), dimethyl acetamide (“DMAC”), dimethyl sulfoxide (“DMSO”), dimethylformamide (“DMF”), N-methyl-2-pyrrolidone, or a mixture thereof. The chemical reticulation may be followed by washing or drying.

In one embodiment, combustion reticulation may be employed in which a combustible atmosphere, e.g., a mixture of hydrogen and oxygen or methane and oxygen, is ignited, e.g., by a spark. In another embodiment, combustion reticulation employs a mixture of hydrogen, oxygen and/or nitrogen.

Combustion reticulation is conducted in a pressure chamber. In an exemplary procedure, the pressure in the pressure chamber is substantially reduced from atmospheric conditions, e.g., to below about 50 -150 millitorr by evacuation for at least about 2 minutes, before, e.g., hydrogen, oxygen or a mixture thereof, is introduced. In another embodiment, the pressure in the pressure chamber is substantially reduced in more than one cycle, e.g., the pressure is substantially reduced, an unreactive gas such as argon or nitrogen is introduced then the pressure is again substantially reduced, before hydrogen, oxygen or a mixture thereof is introduced. The temperature at which reticulation occurs can be influenced by, e.g., the temperature at which the chamber is maintained and/or by the hydrogen/oxygen ratio in the chamber. In another embodiment, combustion reticulation is followed by an annealing period.

### Forming Grafts

An exemplary method of forming a graft embodiment will now be explained, with reference to FIGS. 3-4 and 7. In an embodiment, the graft 10 is made by first obtaining a film layer 20 in the shape of a tube, which film will form the inner surface 15 of the graft. An example of such a procedure is disclosed in U.S. Patent Application Publication No. 2017/0360584, which is incorporated by reference herein in its entirety. To form the film into the shape of a tube, the film is cut to a selected size and wrapped around the outside diameter of a mandrel 19, as shown in FIG. 4. Preferably, the mandrel 19 is first wrapped in a release layer (not shown), such as helically wrapped ePTFE tape, in order to facilitate removal of the tube 12 from the mandrel 19. A single layer or multiple layers of the film may be used to achieve the desired thickness of the inner film layer 20. The mandrel 19 may have a constant or variable outer diameter to create tubes of various geometries such a tapered tube. The film may be treated, such as wet with isopropyl alcohol, to enable the film to lay more smoothly.

Next, the desired size, spacing, and positioning of the foam material 40 is chosen, and the foam material 40 is positioned over the inner film layer 40 at the desired locations. The foam material 40 and/or separate foam members 50, 52, 54 may be pre-cut to an appropriate size before using the foam material 40 in the manufacturing process. An outer film layer 24 is placed over one or more covered portions 42 of the foam material 40, leaving the foam material 40 exposed at the one or more exposed portions 44. The foam material 40 and the outer film layer 24 may be secured to the mandrel 19, such as with wire, wrapping it in ePTFE tape, or using heat-shrink tubing. The outer film layer 24 may be formed similarly to the inner film layer 20, such as by wrapping the outer film material around the foam material 40 and the inner layers (e.g., the film layers 20, 22 and reinforcing element 30) or placing layers of the film material over the foam material 40 and inner layers in a plurality of layers, and connecting the layers together. The outer film layer 24 in this embodiment will form the outer surface 17 of the graft 10 at the one or more covered portions 42 after lamination, whereas the foam material will form the outer surface 17 of the graft 10 at the one or more exposed portions 44. In a configuration where a portion of the foam material 40 extends beyond the film layers 20, 22, 24 at the ends 13, 14 of the tube 12, the foam material 40 may be placed in a configuration such that a portion of the foam material 40 overlaps the inner film layer 20 and a portion of the foam material 40 extends beyond the inner film layer 20, as shown in FIG. 3. A similar placement of the foam material 40 for the embodiment of FIGS. 5-6 is shown in FIG. 6. The foam material 40, or each separate foam member 50, 52, 54 may be provided in the form of a single piece in one embodiment, such as a foam collar that wraps around the outer surface of the inner film layer 20. FIG. 4 illustrates such a foam member 50 in a collar form.

This process may be modified in various ways to result in various configurations. As described above, in some embodiments, the graft 10 includes a reinforcing element 30, such as a stent. In such an embodiment, the reinforcing element 30 may be placed on the mandrel 19 in an appropriate location, such as outside of the inner film layer 20, during the manufacturing process. As shown in FIG. 4, the reinforcing element 30 is placed in contact with the outer surface of the inner film layer 20, and a middle film layer 22 is then placed in a position to partially or completely cover the reinforcing element 30. The middle film layer 22 may be formed similarly to the inner film layer 20, such as by wrapping the middle film material around the reinforcing element 30 and any other inner layers (e.g., the inner film layer 20) or placing layers of the film material over the reinforcing element 30 and other inner layers in a plurality of layers, and connecting the layers together. In this configuration, the foam material 40 is connected to the outer surface of the middle film layer 22, as shown in FIG. 4. Multiple reinforcing elements 30 could be used to allow for the desired flexibility or in the event that corrugations will be added to the graft as described herein. The foam material 40 and any outer layers such as the outer film layer 24, are typically then laminated.

Next, the tube 12 is formed on the mandrel 19, for example, by bonding or otherwise connecting multiple layers of film together to form a single tube 12. This step will simultaneously laminate the foam material 40 between the inner or middle film layer 20, 22 and the outer film layer 24 at the covered portions, and connect the outer film to inner layers (e.g., the inner or middle film layer 20, 22, or the reinforcing element 30) where no foam material 40 is present. The foam material 40 may undergo compression at the covered portions 42 during this step, resulting in the foam material 40 having lower porosity at the covered portions 42 than at the exposed portions 44. Connection may be achieved by heating, such as in an air convection oven set at 150° C. for 10 minutes. Once cool, the tube 12 can be obtained by removing any inner and outer layers used in the process, such as an ePTFE tape release layer or any element used to secure the inner film layer 20 in place on the mandrel. In the case of a microporous UHMWPE film, the time and temperature should be chosen such that the porous nature of the film is not destroyed by thermal degradation. A suitable temperature and time combination may be chosen based on the specific character of the material.

In the case that a corrugation is formed in the tube 12, this may be done as follows. The tube 12 formed above is positioned on a mandrel 19 of sufficient outer diameter such that the inner diameter of the tube fits snugly on the mandrel. In the case that an inner protective layer is used in the previous step, such as the helically wrapped ePTFE tape, the mandrel 19 in this step should have a greater diameter than the mandrel used in the previous step. For each corrugated section, two consecutive sections of the tube are pressed toward each other on the mandrel, such as by hand pressure, thereby buckling the film. The sections are pushed toward each other to create a plurality of corrugations and until strong resistance to further compression of the corrugated section is observed. The procedure is repeated at all other locations where a corrugated section is desired.

Finally, the graft 10 is heat-treated to set the corrugations in the film. In an embodiment, the graft 10 is merely covered with a shrink tube of appropriate size to protect the assembly and placed into a pre-heated oven, such as at 127° C. for 10 min. Another option would be to use a heat gun pre-treatment to partially “lock” the assembly in place. Afterwards, the assembly is removed from the oven, cooled, and then the graft 10 is obtained by removing it from the mandrel 19.

The reinforcing element 30 is connected to the film layers 20, 22, 24 such that the reinforcing element 30 is substantially immobile relative to the film layers 20, 22, 24. The step of connecting is typically carried out by thermal lamination (i.e. heat bonding). The step of connecting the reinforcing element 30 may be performed at the same time as the thermal lamination step used to form the film layers 20, 22, 24. Thus, multiple layers of film may be supplied and the one or more reinforcing elements positioned on the layers of film and then the thermal lamination step carried out in order to both form the film and connect the reinforcing element to the film. An outer film layer 24 covering both the reinforcing element(s) 30 and the inner or middle film layer 20, 22 may also be connected to the reinforcing element(s) 30 and the inner or middle film layer 20, 22. In an embodiment, the reinforcing element(s) 30 is positioned between two film layers (e.g., middle and outer film layers 22, 24) and the thermal lamination connects the three elements together.

In an embodiment where the tube 12 includes one or more second sections 62 devoid of a reinforcing element 30, the second section 62 comprises a modification of the film configured to increase the radial strength and/or the flexibility of the tube 12 in the second section 60. In an embodiment, the second section 60 consists essentially of or consists of the film layers 20, 22, 24. In an embodiment, the second section 62 further comprises a helical wire that may extend across both the first and second sections 60, 62.

Various embodiments of grafts have been described herein, which include various components and features. In other embodiments, the grafts may be provided with any combination of such components and features. It is also understood that in other embodiments, the various devices, components, and features of the grafts described herein may be constructed with similar structural and functional elements having different configurations, including different ornamental appearances.

The graft 10 disclosed herein may have numerous applications in the body, such as in the cardiovascular and neurovascular spaces. The graft 10 may be used in, for example, the treatment of peripheral artery disease in a superficial femoral artery. In an embodiment, the graft has an outer diameter of from 1 or 2 to 10, 9 or 8 mm. In an embodiment, the tube has an outer diameter of from 1 or 2 to 10, 9 or 8 mm. The foam material 40 provides numerous benefits as described herein, and the use of materials such as UHMWPE for the film layers 20, 22, 24 is particularly compatible with the foam materials 40 described herein. These materials provide a lamination temperature that is sufficiently low to permit lamination while not degrading the foam material 40 during manufacturing. For example, the lamination temperature for an UHMWPE membrane is typically below 150° C., while and typical thermoplastic polyurethane (TPU) processing temperatures are >190° C. The temperature that would result in obvious plastic deformation/viscous flow for a slightly crosslinked foam material as described herein would not be reached when heating at 150° C., but may be reached at higher lamination temperatures, e.g., greater than 190° C. or 200° C. Still further benefits and advantages provided by the grafts described herein are apparent to those skilled in the art.

The following non-limiting and non-exhaustive description of exemplary embodiments is intended to further describe certain embodiments of the invention.
1. A graft comprising a tube comprising an inner film layer, an outer film layer disposed radially outward of the inner film layer, and a foam material disposed radially outward of the inner film layer, the outer film layer covering a covered portion of the foam material that is disposed between the outer film layer and the inner film layer, the foam material further comprising an exposed portion that is not covered by the outer film layer.
2. The graft according to any one of the preceding embodiments, wherein the tube further comprises a reinforcing element disposed between the inner film layer and the outer film layer.
3. The graft according to any one of the preceding embodiments, wherein the tube further comprises a middle film layer disposed between the outer film layer and the inner film layer, wherein the reinforcing element is laminated between the middle film layer and the inner film layer.
4. The graft according to any one of the preceding embodiments, wherein the reinforcing element is a stent.
5. The graft according to any one of the preceding embodiments, wherein the foam material is intertwined with the reinforcing element.
6. The graft according to any one of the preceding embodiments, wherein the tube further comprises a middle film layer disposed between the outer film layer and the inner film layer, wherein the covered portion of the foam material is disposed between the middle film layer and the outer film layer.
7. The graft according to any one of the preceding embodiments, wherein the covered portion of the foam material is radially compressed by the outer film layer, relative to the exposed portion of the foam material.
8. The graft according to any one of the preceding embodiments, wherein the exposed portion of the foam material is positioned at a first end of the tube.
9. The graft according to any one of the preceding embodiments, wherein the exposed portion of the foam material extends axially beyond the outer film layer and the inner film layer at the first end.
10. The graft according to any one of the preceding embodiments, wherein the foam material further has a second covered portion that is covered by the outer film layer and disposed between the outer film layer and the inner film layer and a second exposed portion that is separate from the exposed portion and is not covered by the outer film layer.
11. The graft according to any one of the preceding embodiments, wherein the exposed portion of the foam material is positioned at a first end of the tube and the second exposed portion is positioned at a second end of the tube opposite the first end.
12. The graft according to any one of the preceding embodiments, wherein the exposed portion of the foam material is positioned around an entire outer circumference of the tube.
13. The graft according to any one of the preceding embodiments, wherein the exposed portion of the foam material is discontinuous and is positioned around separate portions of an outer circumference of the tube.
14. The graft according to any one of the preceding embodiments, wherein the exposed portion and the covered portion of the foam material are part of a single piece of the foam material.
15. The graft according to any one of the preceding embodiments, wherein the single piece of the foam material further includes a second covered portion that is covered by the outer film layer and disposed between the outer film layer and the inner film layer, such that the covered portion and the second covered portion are on opposite sides of the exposed portion.
16. A method of manufacturing a graft, comprising positioning an inner film layer on a cylindrical mandrel, positioning a foam material radially outward of the inner film layer on the cylindrical mandrel, positioning an outer film layer radially outward of the inner film layer on the cylindrical mandrel, such that the outer film layer covers a covered portion of the foam material that is disposed between the outer film layer and the inner film layer, and wherein an exposed portion of the foam material is not covered by the outer film layer, and thermally bonding the inner film layer and the outer film layer together to connect the inner film layer, the foam material, and the outer film layer together to form a tube.
17. A graft comprising a tube having a plurality of layers defining a tubular structure, the plurality of layers comprising one or more inner layers defining an inner surface of the tube, an outer film layer disposed radially outward of the one or more inner layers, and a foam material covering at least a portion of the one or more inner layers, the outer film layer partially covering the foam material, such that the foam material comprises a covered portion that is covered by the outer film layer and disposed between the outer film layer and the one or more inner layers, and an exposed portion that is not covered by the outer film layer.
18. The graft according to any one of the preceding embodiments, wherein the one or more inner layers includes an inner film layer and a reinforcing element disposed radially outward of the inner film layer.
19. The graft according to any one of the preceding embodiments, wherein the one or more inner layers further includes a mid-film layer disposed between the reinforcing element and the foam material.
20. A graft comprising a tube comprising an inner film layer, an outer film layer, and a foam material between the inner film layer and the outer film layer, wherein the tube comprises one or more covered portions, wherein the foam material is covered by the outer film layer and inner film layer, and one or more exposed portions, wherein the foam material is not covered by the outer film layer.
21. The graft according to any one of the preceding embodiments, wherein the graft comprises 1, 2, 3, 4, 5, 6, 7, 8, 9, or 10 pieces of foam material.
22. The graft according to any one of the preceding embodiments, wherein at the one or more exposed portions the foam material is covered by the inner film on its interior surface.
23. The graft according to any one of the preceding embodiments, wherein the inner film layer comprises the inner surface of the tube.
24. The graft according to any one of the preceding embodiments, wherein the outer film layer comprises the outer surface of the tube.
25. The graft according to any one of the preceding embodiments, wherein the outer film layer laminates the foam material to the tube.
26. The graft according to any one of the preceding embodiments, wherein the inner film layer and/or outer film layer is connected to the foam material.
27. The graft according to any one of the preceding embodiments, wherein the foam material is not covered by either the inner or outer film layer at the exposed portions.
28. The graft according to any one of the preceding embodiments, wherein the outer surface of the foam material is porous at the exposed portions.
29. The graft according to any one of the preceding embodiments, wherein the graft is fenestrated.
30. The graft according to any one of the preceding embodiments, wherein the graft is fenestrated and an exposed portion is present at the connection between a branch and the tube.
31. The graft according to any one of the preceding embodiments, wherein the inner film layer and the outer film layer laminate the foam material between the inner film layer and the outer film layer.
32. The graft according to any one of the preceding embodiments, further comprising a reinforcing element between the inner film layer and the outer film layer.
33. The graft according to any one of the preceding embodiments, wherein the reinforcing element is connected to the inner film layer and/or the outer film layer.
34. The graft according to any one of the preceding embodiments, further comprising a mid-film layer, between the inner film layer and the outer film layer, wherein the mid-film layer and the outer film layer laminate the foam material between the mid-film layer and the outer film layer.
35. The graft according to any one of the preceding embodiments, further comprising a mid-film, between the inner film and the outer film, wherein the inner film and the mid-film laminate the reinforcing element between the inner film and the mid-film.
36. The graft according to any one of the preceding embodiments, further comprising a mid-film layer, between the inner film layer and the outer film layer, wherein the mid-film layer and the outer film layer laminate the foam material between the mid-film layer and the outer film layer.
37. The graft according to any one of the preceding embodiments, wherein the tube comprises at least two exposed portions along the length of the tube.
38. The graft according to any one of the preceding embodiments, wherein the graft comprises a first exposed portion at a first end of the tube.
39. The graft according to any one of the preceding embodiments, wherein the graft comprises a first exposed portion at a first end of the tube and a second exposed portion at a second end of the tube.
40. The graft according to any one of the preceding embodiments, wherein the graft comprises a first covered portion at a first end of the tube.
41. The graft according to any one of the preceding embodiments, wherein the graft comprises a first covered portion at a first end of the tube and a second covered portion at a second end of the tube.
42. The graft according to any one of the preceding embodiments, wherein the graft comprises an exposed portion between a first end of the tube and a second end of the tube.
43. The graft according to any one of the preceding embodiments, wherein the foam material comprises one or more holes within which a first film layer is laminated to a second film layer.
44. The graft according to any one of the preceding embodiments, wherein the foam material comprises one or more holes within which a first film layer is laminated to a second film layer, wherein the first film layer is the inner film layer and the second film layer is the outer film layer.
45. The graft according to any one of the preceding embodiments, wherein the foam material comprises one or more holes within which a first film layer is laminated to a second film layer, wherein the first film layer is the mid-film layer and the second film layer is the outer film layer.
46. The graft according to any one of the preceding embodiments, wherein the foam material comprises one or more holes within which a first film layer is laminated to a second film layer, wherein the first film layer is the inner film layer and the second film layer is the mid-film layer.
47. The graft according to any one of the preceding embodiments, wherein the foam material comprises at least one non-straight edge over which the foam is laminated between a first film layer and a second film layer.
48. The graft according to any one of the preceding embodiments, wherein the outer diameter of the foam material is substantially uniform at a first exposed portion.
49. The graft according to any one of the preceding embodiments, wherein the outer diameter of the foam material is substantially uniform at a second exposed portion.
50. The graft according to any one of the preceding embodiments, wherein the thickness of the foam material is substantially uniform at a first exposed portion.
51. The graft according to any one of the preceding embodiments, wherein the thickness of the foam material is substantially uniform at a second exposed portion.
52. The graft according to any one of the preceding embodiments, wherein the graft comprises a first exposed portion at a first end of the tube and wherein the outer diameter of the foam material increases axially as the distance from the axial midpoint of the tube increases.
53. The graft according to any one of the preceding embodiments, wherein the graft comprises a second exposed portion at a second end of the tube and wherein the outer diameter of the foam material increases as the axial distance from the axial midpoint of the tube increases.
54. The graft according to any one of the preceding embodiments, wherein the foam material is present along the entire outer circumference of the tube at a first exposed portion.
55. The graft according to any one of the preceding embodiments, wherein the foam material is present along a part of but not the entire outer circumference of the tube at a first exposed portion.
56. The graft according to any one of the preceding embodiments, wherein the foam material is in contact with the inner film and the outer film.
57. The graft according to any one of the preceding embodiments, wherein the foam material is in contact with the inner film and the mid-film.
58. The graft according to any one of the preceding embodiments, wherein the foam material is in contact with the mid-film and the outer film.
59. The graft according to any one of the preceding embodiments, wherein the foam material is intertwined with a reinforcing element.
60. The graft according to any one of the preceding embodiments, wherein the foam material comprises a polyurethane foam.
61. The graft according to any one of the preceding embodiments, wherein the polyurethane foam is an open cell polyurethane foam.
62. The graft according to any one of the preceding embodiments, wherein the foam material is biodegradable.
63. The graft according to any one of the preceding embodiments, wherein the foam material is biostable.
64. The graft according to any one of the preceding embodiments, wherein the foam material comprises a thermoset polyurethane.
65. The graft according to any one of the preceding embodiments, wherein the foam material comprises a polycarbonate polyurethane.
66. The graft according to any one of the preceding embodiments, wherein the foam material comprises the reaction product of an aromatic diisocyanate, a polycarbonate diol, and a chain extender.
67. The graft according to any one of the preceding embodiments, wherein the foam material is compressed in the covered portion relative to the foam material in the exposed portion.
68. The graft according to any one of the preceding embodiments, wherein the ratio of the porosity of the foam material in the exposed portion to the porosity of the foam material in the covered portion is at least 2:1, at least 3:1, at least 4:1, or at least 5:1.
69. The graft according to any one of the preceding embodiments, wherein the ratio of the porosity of the foam material in the exposed portion to the porosity of the foam material in the covered portion is at most 20:1, at most 15:1, at most 12:1, at most 10:1, or at most 8:1.
70. The graft according to any one of the preceding embodiments, wherein the foam material has a bulk density of from 0.008 g/cc to 0.96 g/cc.
71. The graft according to any one of the preceding embodiments, wherein the foam material has a bulk density of at least 0.008, 0.010, 0.015, 0.020, 0.025, or 0.03 g/cc.
72. The graft according to any one of the preceding embodiments, wherein the foam material has a bulk density of at most 0.96, 0.75, 0.50, 0.40, 0.30, 0.288, 0.25, 0.20, 0.15, 0.12, 0.115, or 0.104 g/cc.
73. The graft according to any one of the preceding embodiments, wherein the foam material has a void space that comprises from about 70% to about 99% of the volume of the foam material in its uncompressed state.
74. The graft according to any one of the preceding embodiments, wherein the foam material has an average cell size of at least 50, 75, 100, 150, 200, 300, 350, or 400 μm.
75. The graft according to any one of the preceding embodiments, wherein the foam material has an average cell size of at most 1000, 950, 900, 850, 800, 750, 700, 650, 600, 550, 500, 450, or 400 μm.
76. The graft according to any one of the preceding embodiments, wherein the foam material has a compressive strength of at least 3, 4, 5, 10, 15, or 20 kPa.
77. The graft according to any one of the preceding embodiments, wherein the foam material has a compressive strength of at most 70, 60, 50, 40, 30, 20, 15, 14, 13, 12, 11, or 10 kPa.
78. The graft according to any one of the preceding embodiments, wherein the foam material has a tensile strength in both the parallel and perpendicular directions of at least 10, 11, 12, 13, 14, 15, 16, 17, 18, 19, 20, 21, 22, 23, 24, or 25 kPa.
79. The graft according to any one of the preceding embodiments, wherein the foam material has an elongation at break in both the parallel and perpendicular directions of at least 100, 110, 120, 130, 140, 150, 16, 170, or 180%.
80. The graft according to any one of the preceding embodiments, wherein the inner film layer, outer film layer, and/or mid-film layer, if any, comprise polyethylene.
81. The graft according to any one of the preceding embodiments, wherein the inner film layer, outer film layer, and/or mid-film layer, if any, comprise polyethylene.
82. The graft of any one of the preceding exemplary embodiments, wherein the inner film layer, outer film layer, and/or mid-film layer, if any, comprise ultra-high molecular weight polyethylene.
83. The graft of any one of the preceding exemplary embodiments, wherein the inner film layer, outer film layer, and/or mid-film layer, if any, consist of ultra-high molecular weight polyethylene.
84. The graft according to any one of the preceding embodiments, wherein the inner film layer, outer film layer, and/or mid-film layer, if any, comprise an ultra-high molecular weight polyethylene membrane.
85. The graft according to any one of the preceding embodiments, wherein the inner film layer, outer film layer, and/or mid-film layer, if any, comprise an ultra-high molecular weight polyethylene membrane.
86. The graft according to any one of the preceding embodiments, wherein the inner film layer, outer film layer, and/or mid-film layer, if any, consist of an ultra-high molecular weight polyethylene membrane.
87. The graft according to any one of the preceding embodiments, wherein the inner film layer, outer film layer, and/or mid-film layer, if any, consist of an ultra-high molecular weight polyethylene membrane.
88. The graft of any one of the preceding exemplary embodiments, wherein the inner film layer, outer film layer, and/or mid-film layer is non-porous.
89. The graft of any one of the preceding exemplary embodiments, wherein the inner film layer, outer film layer, and/or mid-film layer comprises pores.
90. The graft of any one of the preceding exemplary embodiments, wherein the inner film layer, outer film layer, and/or mid-film layer comprises micropores.
92. The graft of any one of the preceding exemplary embodiments, wherein the inner film layer, outer film layer, and/or mid-film layer comprises nanopores.
93. The graft of any one of the preceding exemplary embodiments, wherein the inner film layer, outer film layer, and/or mid-film layer comprises a plurality of layers of ultra-high molecular
93. The graft of any one of the preceding exemplary embodiments, wherein the inner film layer, outer film layer, and/or mid-film layer comprises from 2 to 8 layers of ultra-high molecular weight polyethylene membrane.
94. The graft of any one of the preceding exemplary embodiments, wherein the inner film layer, outer film layer, and/or mid-film layer has a thickness of from 10 μm to 150, 100, 90, 80, 70, or 60 μm.
95. The graft of any one of the preceding exemplary embodiments, wherein the inner film layer, outer film layer, and/or mid-film layer has a thickness of from 15, 20, 30 or 40 μm to 100, 90, 80, 70 or 60 μm.
96. The graft of any one of the preceding exemplary embodiments, wherein the inner film layer, outer film layer, and/or mid-film layer has a thickness of from 15 μm to 60 μm.
97. The graft of any one of the preceding exemplary embodiments, wherein the inner film layer, outer film layer, and/or mid-film layer has a lamination temperature in a range having a lower end of 100° C., 110° C., 120° C., 130° C., or 140° C., and an upper end of 140° C., 150° C., 160° C., 170° C., 180° C., 190° C., or 200° C.
98. The graft of any one of the preceding exemplary embodiments, wherein the exposed portion comprises the reinforcing element.
99. The graft of any one of the preceding exemplary embodiments, wherein the reinforcing element comprises a stent.
100. The graft of any one of the preceding exemplary embodiments, wherein the stent comprises a zigzag stent.
101. The graft of any one of the preceding exemplary embodiments, wherein the reinforcing element comprises a helical wire.
102. The graft of any one of the preceding exemplary embodiments, wherein the stent comprises a plurality of cylindrical elements.
103. The graft of any one of the preceding exemplary embodiments, wherein the stent comprises a plurality of cylindrical elements connected by one or more bridges.
104. The graft of any one of the preceding exemplary embodiments, wherein the stent is metal.
105. The graft of any one of the preceding exemplary embodiments, wherein the graft has an outer diameter of from 1 or 2 to 10, 9 or 8 mm.
106. The graft of any one of the preceding exemplary embodiments, wherein the tube has an outer diameter of from 1 or 2 to 10, 9 or 8 mm.
107. The graft of any one of the preceding exemplary embodiments, wherein the tube comprises corrugations set in the inner film layer and the outer film layer.
108. The graft of any one of the preceding exemplary embodiments, wherein the tube comprises a corrugated section wherein the inner and the outer film layers comprise one or more corrugations.
109. The graft of any one of the preceding exemplary embodiments, wherein the tube comprises a corrugated section wherein the inner film layer and/or the outer film layer comprise one or more corrugations, wherein the corrugated section is devoid of reinforcing element.
110. The graft of any one of the preceding exemplary embodiments, wherein the tube comprises a corrugated section wherein the inner film layer and/or the outer film layer comprise one or more corrugations, wherein the corrugated section is devoid of foam material.
111. The graft of any one of the preceding exemplary embodiments, wherein the corrugation is set, fixed, and/or locked in the inner film layer and the outer film layer.
112. The graft of any one of the preceding exemplary embodiments, wherein the modification is set, fixed, and/or locked in the inner film layer and the outer film layer as a result of thermal treatment.
113. The graft of any one of the preceding exemplary embodiments, wherein the film has been thermally treated to set, fix, and/or lock the modification in the film itself.
114. The graft of any one of the preceding exemplary embodiments, wherein modification is formed by buckling and thermally treating the inner film layer and/or the outer film layer.
115. The graft of any one of the preceding exemplary embodiments, wherein the corrugation comprises non-helical undulations of the film.
116. The graft of any one of the preceding exemplary embodiments, wherein the corrugation comprises a plurality of circular undulations.
117. The graft of any one of the preceding exemplary embodiments, wherein the corrugation is formed by compressing the inner film layer and/or the outer film layer axially along the axis of the tube and thermally treating the inner film layer and/or the outer film layer.
118. The graft of any one of the preceding exemplary embodiments, wherein a single piece of film extends across all of the covered sections.
119. The graft of any one of the preceding exemplary embodiments, wherein a single piece of film extends across all of the covered sections and the exposed sections.
120. The graft of any one of the preceding exemplary embodiments, wherein the inner wall of the tube forms the inner diameter of the graft.
121. A method of forming a graft comprising the steps of:
   a. placing an inner film layer on a mandrel;
   b. placing a foam material on the inner film layer;
   c. placing an outer film layer on the foam material and the inner film layer; and
   d. thermally bonding the inner film layer and the outer film layer together to connect the inner film layer, the foam material, and the outer film layer together, thereby forming a tube;
   wherein the tube comprises a covered portion, wherein the foam material is covered by the outer film layer and the inner film layer, and an exposed portion, wherein the foam material is not covered by the outer film layer.
122. A method of forming a graft comprising the steps of:
   a. placing an inner film layer on a mandrel;
   b. placing a foam material on the inner film layer;
   c. placing an outer film layer on the foam material such that part of the outer film layer is over the foam material and part of the outer film layer is over the inner film layer and not over the foam material, thereby creating a covered portion, wherein the foam material is covered by the outer film layer, and an exposed portion, wherein the foam material is not covered by the outer film layer; and
   d. thermally bonding the inner film layer and the outer film layer together to connect the inner film layer, the foam material, and the outer film layer together, thereby forming a tube.
123. A method of forming a graft comprising the steps of:
   a. placing an inner film layer on a mandrel;
   b. placing a foam material on the inner film layer;
   c. placing an outer film layer on the foam material such that part of the outer film layer is over the foam material and part of the outer film layer is over the inner film layer and not over the foam material, thereby creating a covered portion, wherein the foam material is covered by the outer film layer, and an exposed portion, wherein the foam material is not covered by the outer film layer; and
   d. thermally bonding the inner film layer and the outer film layer together to connect the inner film layer, the foam material, and the outer film layer together, thereby forming a tube.
124. A method of forming a graft comprising the steps of:
   a. placing an inner film layer on a mandrel;
   b. placing a foam material on the inner film layer such that the foam material is over the inner film layer at a first location and extends beyond the inner film layer at a second location;
   c. placing an outer film layer on the foam material and inner film layer, such that the outer film layer is aligned with the inner film layer, thereby creating a covered portion, wherein the foam material is covered by the inner film layer and the outer film layer, and an exposed portion, wherein the foam material is not covered by the inner film layer or the outer film layer; and
   d. thermally bonding the inner film layer and the outer film layer together to connect the inner film layer, the foam material, and the outer film layer together, thereby forming a tube.
125. A method of forming a graft comprising the steps of:
   a. providing a first film present on a mandrel;
   b. placing a foam material on the first film such that the foam material is over the first film at a first location and extends beyond the first film at a second location;
   c. placing a second film on the foam material and first film, thereby creating a covered portion, wherein the foam material is covered by the first film and the second film, and an exposed portion, wherein the foam material is not covered by at least the second film; and
   d. thermally bonding the first film and the second film together to connect the first film, the foam material, and the second film together, thereby forming a tube.
126. The method of the previous exemplary embodiment, wherein the film is an inner film layer or a mid-film layer, and the second film is a mid-film layer or an outer film layer.
127. The method of any one of the previous exemplary embodiments, further comprising the step of placing a reinforcing element on the inner film layer and/or the foam material prior to placing the outer film layer.
128. A method of forming a graft comprising the steps of:
   a. placing an inner film layer on a mandrel;
   b. placing a foam material on the inner film layer such that the foam material is over the inner film layer at a first location and extends beyond the inner film layer at a second location;
   c. placing a mid-film layer on the foam material;
   d. placing a reinforcing element on the mid-film layer;
   e. placing an outer film layer on the reinforcing element, thereby creating a covered portion, wherein the foam material is covered by the inner film layer, mid-film layer, and the outer film layer, and an exposed portion, wherein the foam material is not covered by the inner film layer, mid-film layer, or the outer film layer; and
   f. thermally bonding the inner film layer and the outer film layer together to connect the inner film layer, the foam material, and the outer film layer together, thereby forming a tube.
129. A method of forming a graft comprising the steps of:
   a. placing an inner film layer on a mandrel;
   b. placing a reinforcing element on the inner film layer;
   c. placing a mid-film layer on the reinforcing element;
   d. placing a foam material on the mid-film layer such that the foam material is over the mid-film layer at a first location and extends beyond the mid-film layer at a second location;
   e. placing an outer film layer on the foam material, thereby creating a covered portion, wherein the foam material is covered by the inner film layer, mid-film layer, and the outer film layer, and an exposed portion, wherein the foam material is not covered by the inner film layer, mid-film layer, or the outer film layer; and
   f. thermally bonding the inner film layer and the outer film layer together to connect the inner film layer, the foam material, and the outer film layer together, thereby forming a tube.
130. The method of any one of the preceding exemplary embodiments, further comprising the steps of compressing the tube at one or more locations, thereby forming a corrugation, and thermally treating the tube to set the corrugations.
131. The method of any one of the preceding exemplary embodiments, wherein the tube is the tube of the graft according to any one of the previous exemplary embodiments.
132. The method of any one of the preceding exemplary embodiments, further comprising the step of bonding a plurality of layers to form any of the inner film layer, outer film layer, or mid-film layer.
133. The method of any one of the preceding exemplary embodiments, wherein the step of thermally bonding is performed at a lamination temperature of the one or more film layers.
134. The method of any one of the preceding exemplary embodiments, wherein the step of thermally bonding is performed at a lamination temperature of the one or more film layers.
135. The method of any one of the preceding exemplary embodiments, wherein the step of thermally bonding at a temperature below a degradation temperature of the foam material.
136. The method of any one of the preceding exemplary embodiments, wherein the step of thermally bonding is performed at a lamination temperature of the one or more film layers and the lamination temperature is below a degradation temperature of the foam material.
137. The method of any one of the preceding exemplary embodiments, wherein the inner film layer, the outer film layer, and the foam material are heated to a temperature in a range having a lower end of 100° C., 110° C., 120° C., 130° C., or 140° C., and an upper end of 140° C., 150° C., 160° C., 170° C., 180° C., 190° C., or 200° C.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “plurality,” as used herein, indicates any number greater than one, either disjunctively or conjunctively, as necessary, up to an infinite number. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

1. A graft comprising:
a tube comprising:
an inner film layer;
an outer film layer disposed radially outward of the inner film layer; and
a foam material disposed radially outward of the inner film layer, the outer film layer covering a covered portion of the foam material that is disposed between the outer film layer and the inner film layer, the foam material further comprising an exposed portion that is not covered by the outer film layer.

2. The graft of claim 1, wherein the tube further comprises a reinforcing element disposed between the inner film layer and the outer film layer.

3. The graft of claim 2, wherein the tube further comprises a middle film layer disposed between the outer film layer and the inner film layer, wherein the reinforcing element is laminated between the middle film layer and the inner film layer.

4. The graft of claim 2, wherein the reinforcing element is a stent.

5. The graft of claim 2, wherein the foam material is intertwined with the reinforcing element.

6. The graft of claim 1, wherein the tube further comprises a middle film layer disposed between the outer film layer and the inner film layer, wherein the covered portion of the foam material is disposed between the middle film layer and the outer film layer.

7. The graft of claim 1, wherein the covered portion of the foam material is radially compressed by the outer film layer, relative to the exposed portion of the foam material.

8. The graft of claim 1, wherein the exposed portion of the foam material is positioned at a first end of the tube.

9. The graft of claim 8, wherein the exposed portion of the foam material extends axially beyond the outer film layer and the inner film layer at the first end.

10. The graft of claim 1, wherein the foam material further has a second covered portion that is covered by the outer film layer and disposed between the outer film layer and the inner film layer and a second exposed portion that is separate from the exposed portion and is not covered by the outer film layer.

11. The graft of claim 10, wherein the exposed portion of the foam material is positioned at a first end of the tube and the second exposed portion is positioned at a second end of the tube opposite the first end.

12. The graft of claim 1, wherein the exposed portion of the foam material is positioned around an entire outer circumference of the tube.

13. The graft of claim 1, wherein the exposed portion of the foam material is discontinuous and is positioned around separate portions of an outer circumference of the tube.

14. The graft of claim 1, wherein the exposed portion and the covered portion of the foam material are part of a single piece of the foam material.

15. The graft of claim 14, wherein the single piece of the foam material further includes a second covered portion that is covered by the outer film layer and disposed between the outer film layer and the inner film layer, such that the covered portion and the second covered portion are on opposite sides of the exposed portion.

16. A method of manufacturing a graft, comprising:
positioning an inner film layer on a cylindrical mandrel;
positioning a foam material radially outward of the inner film layer on the cylindrical mandrel;
positioning an outer film layer radially outward of the inner film layer on the cylindrical mandrel, such that the outer film layer covers a covered portion of the foam material that is disposed between the outer film layer and the inner film layer, and wherein an exposed portion of the foam material is not covered by the outer film layer; and
thermally bonding the inner film layer and the outer film layer together to connect the inner film layer, the foam material, and the outer film layer together to form a tube.

17. A graft comprising:
a tube having a plurality of layers defining a tubular structure, the plurality of layers comprising:
one or more inner layers defining an inner surface of the tube;
an outer film layer disposed radially outward of the one or more inner layers; and
a foam material covering at least a portion of the one or more inner layers, the outer film layer partially covering the foam material, such that the foam material comprises a covered portion that is covered by the outer film layer and disposed between the outer film layer and the one or more inner layers, and an exposed portion that is not covered by the outer film layer.

18. The graft of claim 1, wherein the exposed portion of the foam material extends axially beyond the outer film layer at a first end of the tube.

19. The graft of claim 18, wherein a second exposed portion of the foam material extends axially beyond the outer film layer at a second end of the tube, wherein the second end is opposite the first end.

20. The graft of claim 19, wherein the foam material at the exposed portion and the foam material at the second exposed portion are two separate pieces of foam material.
